SEQUENCE LISTING (55 sequences)

SEQ ID NO: 1 - protein, 388 aa, Artificial
  Consensus apoL-I
MEGAALLRLFVLCIWTSGLFLGVGVRAEEATARVQQNHPGGTDTGDPQESNVSLEDYLNYFQKNVSPQEMLLLLSDHKAWERVVATAELPRDEADELYKALNKLIRHMVMKDKNWLEEVQQHRKRFLEEFPRLERELQDKIRRLCDLAGQVQKVHKGATIANAFSSTLGVASGVLTFLGLGLAPFTAGSSLVLLEPVTGLGIAAALTGITSGSVEYAKKRWAQAEAHELVNKSLDTVEEMNEFLYHNIPNFISLRVNLVKFTEDTGKAIRAIRQARANPHSVSHVPASLHRVTEPVSATSVEERARVVEMERVAESRTTEVIRGAKIVDKVFEGALFVLDVVGLVCQLKHLHEGAKSXTAXXLKKVAQXLXXXLXILXXXYXTLRQXP

SEQ ID NO: 2 - protein, 398 aa, Homo sapiens
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 3 - protein, 398 aa, Homo sapiens
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNKKYKILQADQEL

SEQ ID NO: 4 - protein, 396 aa, Artificial
  Consensus apoL-I
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNXXYXTLRQXP

SEQ ID NO: 5 - protein, 388 aa, Papio hamadryas
MEGAALLRLFVLCIWTSGLFLGVGVRAEEATARVQQNHPGGTDTGDPQESNVSLEDYLNYFQKNVSPQEMLLLLSDHKAWERVVATAELPRDEADELYKALNKLIRHMVMKDKNWLEEVQQHRKRFLEEFPRLERELQDKIRRLCDLAGQVQKVHKGATIANAFSSTLGVASGVLTFLGLGLAPFTAGSSLVLLEPVTGLGIAAALTGITSGSVEYAKKRWAQAEAHELVNKSLDTVEEMNEFLYHNIPNFISLRVNLVKFTEDTGKAIRAIRQARANPHSVSHVPASLHRVTEPVSATSVEERARVVEMERVAESRTTEVIRGAKIVDKVFEGALFVLDVVGLVCQLKHLHEGAKSKTAEELKKVAQELEKKLNILNKKYETLRQEP

SEQ ID NO: 6 - protein, 60 aa, Homo sapiens
APVSFFLVLDVVYLVYESKHLHEGAKSETAEELKKVAQELEEKLNILNNNYKILQADQEL

SEQ ID NO: 7 - protein, 58 aa, Papio hamadryas
VFEGALFVLDVVGLVCQLKHLHEGAKSKTAEELKKVAQELEKKLNILNKKYETLRQEP

SEQ ID NO: 8 - protein, 58 aa, Artificial
  apoL-I chimera
APVSFFLVLDVVYLVYESKHLHEGAKSKTAEELKKVAQELEKKLNILNKKYETLRQEP

SEQ ID NO: 9 - protein, 58 aa, Artificial
  apoL-I chimera
APVSFFLVLDVVYLVYESKHLHEGAKSETAEELKKVAQELEKKLNILNKKYETLRQEP

SEQ ID NO: 10 - protein, 58 aa, Artificial
  apoL-I chimera
APVSFFLVLDVVYLVYESKHLHEGAKSETAEELKKVAQELEEKLNILNKKYETLRQEP

SEQ ID NO: 11 - protein, 58 aa, Artificial
  apoL-I chimera
APVSFFLVLDVVYLVYESKHLHEGAKSETAEELKKVAQELEEKLNILNNNYETLRQEP

SEQ ID NO: 12 - protein, 60 aa, Artificial
  apoL-I chimera
APVSFFLVLDVVYLVYESKHLHEGAKSETAEELKKVAQELEEKLNILNKKYKILQADQEL

SEQ ID NO: 13 - protein, 48 aa, Artificial
  Consensus apoL-I C-terminal
VFEGALFVLDVVGLVCQLKHLHEGAKSXTAXXLKKVAQXLXXXLXILX

SEQ ID NO: 14 - protein, 348 aa, Homo sapiens
MSDLGAVISLLLWGRQLFALYSGNDVTDISDDRFPKPPEIANGYVEHLFRYQCKNYYRLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYHQVDIGLIKLKQKVLVNERVMPICLPSKNYAEVGRVGYVSGWGQSDNFKLTDHLKYVMLPVADQYDCITHYEGSTCPKWKAPKSPVGVQPILNEHTFCVGMSKYQEDTCYGDAGSAFAVHDLEEDTWYAAGILSFDKSCAVAEYGVYVKVTSIQHWVQKTIAEN

SEQ ID NO: 15 - DNA, 1245 bp, Homo sapiens
actgctcttccagaggcaagaccaaccaagatgagtgacctgggagctgtcatttccctcctgctctggggacgacagctttttgcactgtactcaggcaatgatgtcacggatatttcagatgaccgcttcccgaagccccctgagattgcaaatggctatgtggagcacttgtttcgctaccagtgtaagaactactacagactgcgcacagaaggagatggagtatacaccttaaatgataagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggggccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggtggttctacaccctaactaccaccaggtagatattgggctcatcaaactcaaacagaaggtgcttgttaatgagagagtgatgcccatctgcctaccttcaaagaattatgcagaagtagggcgtgtgggttacgtgtctggctggggacaaagtgacaactttaaacttactgaccatctgaagtatgtcatgctgcctgtggctgaccaatacgattgcataacgcattatgaaggcagcacatgccccaaatggaaggcaccgaagagccctgtaggggtgcagcccatactgaacgaacacaccttctgtgtcggcatgtctaagtaccaggaagacacctgctatggcgatgcgggcagtgcctttgccgttcacgacctggaggaggacacctggtacgcggctgggatcctaagctttgataagagctgtgctgtggctgagtatggtgtgtatgtgaaggtgacttccatccagcactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccaaaa

SEQ ID NO: 16 - protein, 348 aa, Papio hamadryas
MSDLGAVISLLLWGRQLFALYSGNDVTDIPDDSFLKPPMIANGYVEHLVRYQCKSYYRLRTEGDGVYTLNNEKQWTNKAVGDKLPECEAVCGKPKNPADAVQRILGGHLDAKGSFPWQAKMVSRHNLTTGATLINEQWLLTTAKNLFLNHSENATAKDIAPTLTLYVGKKQLVEIEKVVLHPNYSQVDIGLIKLKQKVPVNERVMPICLPSKDYAEVGRVGYVSGWGRNANFNFTDHLKYVMLPVADQYDCIKHYEGSKCPKRKAPKNPVGVQPILNEHTFCAGMSKYQEDTCYGDAGTAFAVHDMEEDTWYAAGILSFDKSCGVAKYGVYVKATSIQDWVQKTIAEN

SEQ ID NO: 17 - DNA, 1047 bp, Papio hamadryas
atgagtgacctgggagctgtcatttccctcctgctctggggacgacagctttttgcattgtactcaggcaatgatgtcacggatatcccagatgacagcttcctgaagccccctatgatcgcaaatggctacgtggagcacttggttcgctaccagtgtaagagctactacaggctgcgcacagaaggagatggagtgtacaccttaaacaatgagaagcagtggacaaataaggctgttggagataaacttcctgaatgtgaagcagtgtgtgggaagcccaagaatccggcagacgcagtgcagcggatcctgggtggacacctggatgccaaaggcagctttccctggcaggctaagatggtttcccgccataatctcaccacaggggccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggtggttctacaccctaactactcccaggtagatattgggctcatcaaactcaaacagaaggtgcctgttaatgagagagtgatgcccatctgcctaccctcaaaggattatgcagaagtagggcgtgtgggttacgtgtctggctgggggcgaaatgccaattttaactttactgaccatctgaagtatgtcatgctgcctgtggctgaccaatacgattgcataaagcactatgaaggcagcaaatgccccaaaaggaaggcaccgaagaaccctgtaggggtgcagcccatactgaatgaacacaccttctgtgccggcatgtctaagtaccaggaagacacctgctatggcgatgcaggcactgcctttgccgttcacgacatggaggaggacacctggtatgcagctgggatcctaagctttgataagagctgtggtgtggctaagtatggtgtgtatgtgaaggcgacttccatccaggactgggttcagaagaccatagctgagaactaa

SEQ ID NO: 18 - DNA, 18 bp, Artificial
  Primer, SP1
ctccagctcctgagccac

SEQ ID NO: 19 - DNA, 20 bp, Artificial
  Primer, SP2
gctgagactggctcagtgac

SEQ ID NO: 20 - DNA, 37 bp, Artificial
  Primer, Forward
ctcgaggccaccatggagggagctgctttgctgagac

SEQ ID NO: 21 - DNA, 21 bp, Artificial
  Primer, Reverse
ggtggttgccctgccctgtgg

SEQ ID NO: 22 - DNA, 24 bp, Artificial
  Primer, Reverse
gcataatcctttgagggtaggcag

SEQ ID NO: 23 - protein, 7 aa, Papio hamadryas
QKVEPLR

SEQ ID NO: 24 - protein, 7 aa, Papio hamadryas
LHELHEK

SEQ ID NO: 25 - protein, 8 aa, Papio hamadryas
AELHEGTR

SEQ ID NO: 26 - protein, 9 aa, Papio hamadryas
LSPLGEEVR

SEQ ID NO: 27 - protein, 9 aa, Papio hamadryas
AKPALEDLR

SEQ ID NO: 28 - protein, 11 aa, Papio hamadryas
QGLLPVLESFK

SEQ ID NO: 29 - protein, 11 aa, Papio hamadryas
DLVTVYVEALK

SEQ ID NO: 30 - protein, 10 aa, Papio hamadryas
VQPYLDDFQK

SEQ ID NO: 31 - protein, 9 aa, Papio hamadryas
WQEEMELYR

SEQ ID NO: 32 - protein, 11 aa, Papio hamadryas
THLAPYSDELR

SEQ ID NO: 33 - protein, 12 aa, Papio hamadryas
VSFLSALEEYTK

SEQ ID NO: 34 - protein, 13 aa, Papio hamadryas
DYVSQFEGSALGK

SEQ ID NO: 35 - protein, 10 aa, Papio hamadryas
KWQEEMELYR

SEQ ID NO: 36 - protein, 17 aa, Papio hamadryas
DSGKDYVSQFEGSALGK

SEQ ID NO: 37 - protein, 16 aa, Papio hamadryas
EQLGPVTQEFWDNLEK

SEQ ID NO: 38 - protein, 22 aa, Papio hamadryas
EQLGPVTQEFWDNLEKETEGLR

SEQ ID NO: 39 - protein, 7 aa, Papio hamadryas
DYAEVGR

SEQ ID NO: 40 - protein, 8 aa, Papio hamadryas
NPADAVQR

SEQ ID NO: 41 - protein, 8 aa, Papio hamadryas
QKVPVNER

SEQ ID NO: 42 - protein, 8 aa, Papio hamadryas
GSFPWQAK

SEQ ID NO: 43 - protein, 9 aa, Papio hamadryas
ILGGHLDAK

SEQ ID NO: 44 - protein, 9 aa, Papio hamadryas
VGYVSGWGR

SEQ ID NO: 45 - protein, 9 aa, Papio hamadryas
VMPICLPSK

SEQ ID NO: 46 - protein, 10 aa, Papio hamadryas
ATSIQDWVQK

SEQ ID NO: 47 - protein, 13 aa, Papio hamadryas
TEGDGVYTLNNEK

SEQ ID NO: 48 - protein, 15 aa, Papio hamadryas
LRTEGDGVYTLNNEK

SEQ ID NO: 49 - protein, 7 aa, Papio hamadryas
VAQELEK

SEQ ID NO: 50 - protein, 6 aa, Papio hamadryas
KYETLR

SEQ ID NO: 51 - protein, 7 aa, Papio hamadryas
TAEELKK

SEQ ID NO: 52 - protein, 7 aa, Papio hamadryas
FLEEFPR

SEQ ID NO: 53 - protein, 9 aa, Papio hamadryas
VVATAELPR

SEQ ID NO: 54 - protein, 13 aa, Papio hamadryas
VTEPVSATSVEER

SEQ ID NO: 55 - protein, 7 aa, Homo sapiens
VTEPISA

(19) United States
(12) Patent Application Publication
(10) Pub. No.: US 2011/0030078 A1
(43) Pub. Date: Feb. 3, 2011

(54) TRYPANOSOME RESISTANT NON-HUMAN TRANSGENIC ANIMAL

(75) Inventors: Jayne RAPER, Carmel, NY (US); Russell THOMPSON, New York, NY (US); Marie SAMANOVIC, New York, NY (US); Maria Del Pilar Molina PORTELA, New York, NY (US)

Correspondence Address: NIXON PEABODY LLP - PATENT GROUP, 1100 CLINTON SQUARE, ROCHESTER, NY, 14604, US

(73) Assignee: NEW YORK UNIVERSITY, New York, NY (US)

(21) Appl. No.: 12/848,738

(22) Filed: Aug. 2, 2010

Related U.S. Application Data
(60) Provisional application No. 61/230,470, filed on Jul. 31, 2009.

(51) Int. Cl. A01K 67/027; A01K 67/00; C12N 15/873; C12N 5/10

(52) U.S. Cl. 800/15; 800/13; 800/14; 800/18; 800/16; 800/17; 800/21; 435/325

(57) ABSTRACT

The present invention is directed to a Trypanosome-resistant, non-human transgenic animal whose somatic and germ cells comprise a nucleic acid which encodes an apolipoprotein L-I polypeptide (apoL-I). The apoL-I protein has the amino acid sequence selected from the group consisting of SEQ ID NO: 1, SEQ ID NO: 3, SEQ ID NO: 4, and SEQ ID NO: 5. The first nucleic acid transgene is operatively associated with at least one expression regulatory sequence. Methods of producing and raising such transgenic animals as well as transgenic eggs and sperm are also disclosed.

This application claims the benefit of U.S. Provisional Patent Application Ser. No. 61/230,470, filed Jul. 31, 2009, which is hereby incorporated by reference in its entirety.

This invention was made with government support under grant numbers AI-41233 from National Institutes of Health. The government has certain rights in this invention.

### FIELD OF THE INVENTION

The present invention is directed to a Trypanosome resistant non-human transgenic animal.

### BACKGROUND OF THE INVENTION

Trypanosomiasis is ranked among the top 10 global cattle diseases impacting the poor (Perry, “Investing in Animal Health Research to Alleviate Poverty,” pp. 148. International Livestock Research Institute, Nairobi, Kenya (2002)). It is a long standing problem, and it has been estimated that the cost of the disease to livestock keepers and consumers exceeds US$ 1 billion annually (Kristjanson et al., “Measuring the Costs of African Trypanosomosis, the Potential Benefits of Control and Returns to Research,” Agricultural Systems 59:79-98 (1999)). This is one of the most significant diseases to threaten livestock in infected areas and this disease causes immense human suffering through loss of critical livestock which are central to African small scale agriculture (Swallow, “Impacts of Trypanosomiasis on African Agriculture,” pp. 52., Food and Agriculture Organization of the United Nations, Rome, Italy (2000)).

Both humans and livestock are susceptible to this fatal disease. It is caused by the extracellular protozoan parasites Trypanosoma spp. that are transmitted by Tsetse flies (Glossina spp.). Related trypanosomes are responsible for sleeping sickness in humans. There are two human serum resistant sub-species Trypanosoma brucei rhodesiense and Trypanosoma brucei gambiense, both of which have livestock reservoirs. Epidemiological analyses in East Africa reveal that 1-10% of cattle and 4% of pigs carry T. b. rhodesiense (Welburn et al., “Identification of Human-infective Trypanosomes in Animal Reservoir of Sleeping Sickness in Uganda by Means of Serum-resistance-associated (SRA) Gene,” Lancet 358:2017-2019 (2001); Waiswa et al., “Domestic Animals as Reservoirs for Sleeping Sickness in Three Endemic Foci in South-eastern Uganda,” Ann. Trop. Med. Parasitol. 97:149-155 (2003)), whereas in West Africa 14-50% of pigs carry T. b. gambiense (Simo et al., “High Prevalence of Trypanosoma brucei gambiense Group 1 in Pigs from the Fontem Sleeping Sickness Focus in Cameroon,” Vet. Parasitol. 139:57-66 (2006); Jamonneau, v. et al., “Mixed Infections of Trypanosomes in Tsetse and Pigs and their Epidemiological Significance in a Sleeping Sickness Focus of Cote d'Ivoire,” Parasitology 129:693-702 (2004)). The Trypanosoma brucei group consists of T. b. rhodesiense and T. b. gambiense, which cause human African trypanosomiasis (HAT) or sleeping sickness and T. b. brucei, which cannot infect humans or a subset of catarrhine primates due to trypanolytic factors (TLFs) present in their serum (Lugli et al., “Characterization of Primate Trypanosome Lytic Factors,” Mol. Biochem. Parasitol. 138:9-20 (2004); Seed et al., “A Survey for a Trypanocidal Factor in Primate Sera,” J. Protozoology 37:393-400 (1990); Pays et al., “The Trypanolytic Factor of Human Serum,” Nat. Rev. Microbiol. 4:477-86 (2006)).

High-density lipoproteins complexes (HDLs) are formed of characteristic proteins embedded in a phospholipid monolayer. Trypanosomal lytic factors (TLFs) are a subclass of HDLs that show anti-trypanosome activity (Rifkin et al., “Identification of the Trypanocidal Factor in Normal Human Serum: High Density Lipoprotein,” Proc Nat'l Acad Sci USA 75:3450-54 (1978); Hajduk et al., “Lysis of Trypanosoma brucei by a Toxic Subspecies of Human High Density Lipoprotein,” J Biol Chem 264(9):5210-7 (1989); Hajduk et al., “High-density Lipoprotein-mediated Lysis of Trypanosomes,” Parasitol Today 8(3):95-8 (1992)). Human Trypanosomal Lytic Factors (TLF) are pore forming complexes that contain a trypanolytic toxin, apolipoprotein L-I (apoL-I), a hemoglobin binding protein, haptoglobin related protein (Hpr), which forms a high affinity ligand (Hb-Hpr) that facilitates uptake of TLF via a trypanosome receptor and a ubiquitous HDL protein, Apolipoprotein A-I (apoA-I) (Vanhamme et al., “Apolipoprotein L-I is the Trypanosome Lytic Factor of Human Serum,” Nature 422:83-7 (2003); Vanhollebeke et al., “A Haptoglobin-hemoglobin Receptor Conveys Innate Immunity to Trypanosoma brucei in Humans,” Science 320:677-81 (2008)). Human TLFs comprise two minor HDL sub-fractions (TLF1 and TLF2), characterized by the presence of Haptoglobin related protein (Hpr) and the pore forming protein apoL-I (Vanhamme et al., “Apolipoprotein L-I is the Trypanosome Lytic Factor of Human Serum,” Nature 422:83-7 (2003); Lugli et al., “Characterization of Primate Trypanosome Lytic Factors,” Mol. Biochem. Parasitol. 138:9-20 (2004); Raper et al., “Characterization of a Novel Trypanosome Lytic Factor from Human Serum,” Infect. Immun. 6:1910-6 (1999); Nielsen et al., “Haptoglobin-related Protein is a High-affinity Hemoglobin-binding Plasma Protein,” Blood 108:2846-9 (2006)). TLFs are endocytosed by trypanosomes and activated to form membrane pores under acidic lysosomal conditions. This results in ion disregulation that leads to osmotic imbalance, swelling of the parasite, and ultimately lysis (Molina-Portela et al., “Trypanosome Lytic Factor, A Subclass of High-density Lipoprotein, Forms Cation-selective Pores in Membranes,” Mol. Biochem. Parasitol. 144:218-26 (2005); Perez-Morga et al., “Apolipoprotein L-I Promotes Trypanosome Lysis by Forming Pores in Lysosomal Membranes,” Science 309:469-472 (2005)).

Although both human apoL-I and Hpr have been proposed to kill trypanosomes individually in vitro (Shiflett et al., “Human High Density Lipoproteins are Platforms for the Assembly of Multi-component Innate Immune Complexes,” J Biol Chem 280:32578-85 (2005); Perez-Morga et al., “Apolipoprotein L-I Promotes Trypanosome Lysis by Forming Pores in Lysosomal Membranes,” Science 309:469-72 (2005)), only transgenic mice producing human apoL-I are protected from T. b. brucei infection in vivo (Molina-Portela et al., “Distinct Roles of Apolipoprotein Components within the Trypanosome Lytic Factor Complex Revealed in a Novel Transgenic Mouse Model,” J. Exp. Med. 205:1721-8 (2008); Hatada et al., “No Trypanosome Lytic Activity in the Sera of Mice Producing Human Haptoglobin-related Protein,” Mol Biochem Parasitol 119:291-4 (2002)), whereas mice producing human Hpr are unprotected. Hpr, however, increases the specific activity of human TLF in vitro and in vivo (Shiflett et al., “Human High Density Lipoproteins are Platforms for the Assembly of Multi-component Innate Immune Complexes,” J Biol Chem 280:32578-85 (2005)), which is at least in part due to the binding of Hpr-Hemoglobin (Hpr-Hb) to a Haptoglobin-Hemoglobin (Hp-Hb) receptor on the trypanosome cell surface, leading to an increased uptake of apoL-I (Vanhollebeke et al., “A Haptoglobin-hemoglobin Receptor Conveys Innate Immunity to Trypanosoma brucei in Humans,” Science 320:677-81 (2008); Widener et al., “Hemoglobin is a Co-Factor of Human Trypanosome Lytic Factor,” PLoS Pathogens 3, e129 (2007)). However, the human serum resistant parasite Trypanosoma b. rhodesiense escapes lysis by TLF due to serum resistance associated (SRA) protein, which neutralizes apoL-I HDLs.

While it is unclear what role the animal reservoirs play in T. b. gambiense transmission, it is established that the animal reservoir is very important for transmission of T. b. rhodesiense to humans (Fevre et al., “Human African Trypanosomiasis: Epidemiology and Control,” Adv. Parasitol. 61:167-221 (2006)). It has been emphasized by WHO that the key to preventing disease in people is to treat both human and livestock reservoirs. Conventional parasite control measures are based on the use of curative (chemotherapy) and preventive (chemoprophylaxis) drugs. Only three drugs are available—isometamidium chloride, diminazene aceturate and homidium (bromide and chloride). It is currently estimated that between 40-60 million cattle are at risk and 35 million doses of these chemicals are used in Africa each year. Furthermore, trypanocidal drugs that are effective against trypanosomes, pathogenic to both humans and livestock, have been extensively used in animals (35 million doses/year), which has led to selection of resistant trypanosome strains in at least 13 countries. Thus, a novel approach to trypanosomiasis control is needed.

The present invention is directed at overcoming these and other deficiencies in the art.

### SUMMARY OF THE INVENTION

One aspect of the present invention is related to a Trypanosome-resistant, non-human transgenic animal whose somatic and germ cells comprise a first nucleic acid transgene which encodes an apolipoprotein L-I polypeptide (apoL-I). The apoL-I protein has the amino acid sequence selected from the group consisting of SEQ ID NO: 1, SEQ ID NO: 3, SEQ ID NO: 4, and SEQ ID NO: 5. The first nucleic acid transgene is operatively associated with at least one expression regulatory sequence.

Another aspect of the present invention is related to a method of producing a Trypanosome-resistant, non-human transgenic animal. This method comprises providing a first nucleic acid construct. This first nucleic acid construct has a first nucleic acid molecule which encodes for apolipoprotein L-I polypeptide having the amino acid sequence selected from the group consisting of SEQ ID NO: 1, SEQ ID NO: 3, SEQ ID NO: 4, and SEQ ID NO: 5. The first nucleic acid molecule is operably associated with at least one expression regulatory sequence. The method further involves introducing the first nucleic acid construct into a zygote of a non-human animal and then transplanting the zygote into a pseudopregnant non-human female animal. This transplanted zygote is allowed to develop to term and at least one transgenic offspring containing the first nucleic acid molecule is subsequently identified.

Another aspect of this invention relates to a method for producing a Trypanosome-resistant, non-human transgenic animal. A first nucleic acid construct is provided which is comprised of a first nucleic acid molecule encoding an apolipoprotein L-I polypeptide having the amino acid sequence selected from the group consisting of SEQ ID NO: 1, SEQ ID NO: 3, SEQ ID NO: 4, and SEQ ID NO: 5. This first nucleic acid molecule is operably associated with at least one expression regulatory sequence. The first nucleic acid construct is then introduced into an embryo of a non-human animal and the embryo is transplanted into a pseudopregnant non-human female animal. The embryo is allowed to develop to term, and a transgenic offspring containing the first nucleic acid molecule is subsequently identified.

Another aspect of the present invention is directed to a transgenic egg comprising a first nucleic acid transgene which encodes an apolipoprotein L-I polypeptide having the amino acid sequence selected from the group consisting of SEQ ID NO: 1, SEQ ID NO: 3, SEQ ID NO: 4, and SEQ ID NO: 5. This apolipoprotein L-I encoding nucleic acid transgene is operatively associated with at least one expression regulatory sequence.

Another aspect of the present invention is directed to a transgenic sperm comprising a first nucleic acid transgene which encodes an apolipoprotein L-I polypeptide having the amino acid sequence selected from the group consisting of SEQ ID NO: 1, SEQ ID NO: 3, SEQ ID NO: 4, and SEQ ID NO: 5 and is operatively associated with at least one expression regulatory sequence.

Another aspect of the present invention is directed to a method of animal husbandry where Trypanosome-resistant, non-human transgenic animals are provided and raised.

Human TLFs cannot kill certain subspecies of Trypanosoma brucei such as T. b. rhodesiense or T. b. gambiense, which are both pathogenic to humans, but it can kill Trypanosoma brucei brucei. The human serum resistant parasite Trypanosoma brucei rhodesiense escapes lysis by TLFs due to serum resistance protein (SRA), which neutralizes apoL-I (Xong et al., “A VSG Expression Site-associated Gene Confers Resistance to Human Serum in Trypanosoma rhodesiense,” Cell 95:839-46 (1998), which is hereby incorporated by reference in its entirety). The resistance to human serum by T. b. rhodesiense is due to the production of the SRA protein, which stoichiometrically binds apoL-I and either neutralizes its activity directly in the lysosome (Vanhamme et al., “Apolipoprotein L-I is the Trypanosome Lytic Factor of Human Serum,” Nature 422: 83-87 (2003), which is hereby incorporated by reference in its entirety) and/or re-routes its trafficking away from the lysosome (Oli et al., “Serum Resistance-associated Protein Blocks Lysosomal Targeting of Trypanosome Lytic Factor in Trypanosoma brucei,” Eukaryot Cell 5: 132-139 (2006), which is hereby incorporated by reference in its entirety). The mechanism of resistance in T. b. gambiense is unknown but does not involve SRA.

In order to test for trypanosomal lytic activity, transgenic mice were created using a hydrodynamic gene delivery (HGD) which allowed for simultaneous liver expression of three genes, apoA-I, apoL-I and Hpr, which are components of the TLFs. Contrary to expectations, triple transgenic mice carrying a truncated apoL-I gene devoid of serum resistance gene interacting domain (C-terminal α-helix) were unable to kill any Trypanosoma spp. (Molina-Portela et al., “Distinct Roles of Apolipoprotein Components Within the Trypanosome Lytic Factor Complex Revealed in a Novel Transgenic Mouse Model,” J Exp Med 205:1721-8 (2008), which is hereby incorporated by reference in its entirety).

The present invention discloses that baboons have a TLF complex that contains orthologs of Hpr and apoL-I and that full-length baboon apoL-I confers trypanolytic activity to mice and when expressed together with baboon Hpr and human apoA-I, provides protection against both animal and human serum resistant trypanosomes in vivo. In contrast to humans and gorillas; serum from baboons (Papio spp.) sooty mangabeys (Cercocebus spp.), and mandrills (Mandrillus spp.) can kill both T. b. brucei and T. b. rhodesiense (Lugli et al., “Characterization of Primate Trypanosome Lytic Factors,” Mol Biochem Parasitol 138: 9-20 (2004), which is hereby incorporated by reference in its entirety), via a trypanolytic component found within HDLs.

Surprisingly, and contrary to previous studies, full length baboon apoL-I conferred trypanolytic activity to murine serum and together with baboon Hpr and human apoA-I conferred protection against both animal and human infective trypanosomes in vivo. A previous study of trypanolytic activity of serum from various primates had shown that baboon (Papio hamadryas) serum lacks the lytic ability as well as the apoL-I protein (Poelvoorde et al., “Distribution of Apolipoprotein L-I and Trypanosome Lytic Activity among Primate Sera,” Mol Biochem Parasitol 134:155-157 (2004), which is hereby incorporated by reference in its entirety).

Transgenic mice expressing baboon TLF protein apolipoprotein L-I are completely resistant to T. b. brucei and human serum resistant (infective) T. b. rhodesiense. Baboons (Papio hamadryas) have TLFs that contain orthologues of human Hpr and apoL-I, with significant differences. Baboon apoL-I is 60% identical to human apoL-I at the amino acid level. It is the differences in sequence that govern the ability to kill human serum resistant (infective) trypanosomes (T. b. rhodesiense or T. b. gambiense). Further two critical lysines near the C-terminus of baboon apoL-1 are necessary and sufficient to prevent binding to SRA and thereby confer resistance to human infective trypanosomes. These findings form the basis for the creation of TLF transgenic livestock that would be resistant to animal and human infective trypanosomes, which would result in the reduction of disease and the zoonotic transmission of human infective trypanosomes.

The present invention relates to a method of engineering transgenic (Tg) animals that have innate resistance to trypanosomes. Livestock that do not have TLFs are susceptible to parasitic Trypanosoma spp. Animals engineered to express the TLFs of the present invention will have the capacity to kill a variety of subspecies of trypanosomes thereby reducing disease related mortality as well as the risk to humans.

### BRIEF DESCRIPTION OF THE DRAWINGS

FIGS. 1A-C show the survival kinetics of mice infected with T. b. brucei ILTat1.25, relative lytic units, and protein expression in Tg mice. FIG. 1A shows results for mice expressing Hpr (black diamond; n=13), apoL-I (black square; n=13), or a combination of Hpr and apoL-I in individual plasmids (white triangle; Hpr/apoL-I; n=10) or in a single plasmid (black circle, Hpr:apoL-I; n=6) that were infected i.p. with 1.78×10⁶ trypanosomes. Control mice were treated with empty vector (gray circle; n=14). The parasitemia was monitored and time of death was recorded. FIG. 1B shows survival kinetics of naive mice infected with T. b. brucei ILTat1.25 that were administered plasma i.v. from mice transfected with empty vector (gray circle; n=7), apoL-I (black square; n=7), or a combination of Hpr and apoL-I in a single plasmid (black circle; Hpr:apoL-I; n=3). The protection obtained by normal human plasma (dilution 1/8) is indicated by the inverted triangle. FIG. 1C shows western blot with serial dilutions of plasma from Tg mice expressing murine apoA-I and human Hpr and apoL-I from the same plasmid (Hpr:apoL-I) and Tg mice expressing human apoA-I and human Hpr and apoL-I from the same plasmid (Hpr:apoL-I). Hpr and apoL-I were detected with monoclonal antibodies.

FIGS. 2A-E show increased specific activity of Tg-HDL in the presence of human apoA-I and Hpr. FIG. 2A shows western blot with serial dilutions of plasma from Human Apolipoprotein A-I (HuapoA-I) mice expressing Hpr and apoL-I from a single plasmid (HuapoA-I:Hpr:apoL-I) and normal human plasma. Hpr and apoL-I were detected with monoclonal antibodies. FIG. 2B shows survival kinetics of naive mice infected with T. b. brucei ILTat1.25 and then given 300 μl plasma i.v. from HuapoA-I mice expressing Hpr (black diamond; n=3), apoL-I (black square; n=3), or both Hpr and apoL-I from a single plasmid (black circle, Hpr:apoL-I; n=3). The protection obtained by normal human plasma (dilution 1/8) is indicated by the inverted triangle. FIG. 2C shows A280 profile of KBr-purified lipoproteins from human (dashed line) and HuapoA-I mouse plasma expressing Hpr:apoL-I (red line), Hpr (green line), and apoL-I (blue line) separated by size on a Superdex 200 column; fractions 6-7 are void, fractions 8-9 are human LDL, fractions 10-14 are HDLs, and fraction 15 is albumin. FIG. 2D shows western blot of different size-fractionated lipoproteins from HuapoA-I mice expressing Hpr only (HuapoA-I:Hpr), apoL-I only (HuapoA-I:apoL-I), or Hpr and apoL-I from the same plasmid (HuapoA-I:Hpr:apoL-I). Hpr and apoL-I were detected with monoclonal antibodies. FIG. 2E shows in vitro lytic activity of size-fractionated HDL (fraction 11) from human and HuapoA-I mice expressing Hpr, apoL-I, or both from a single plasmid (dark gray bars). Open bars represent the lytic activity obtained in the presence of NH4Cl (10 mM). Data show the mean and SEM of five independent measurements.

FIGS. 3A-D show that Tr-apoL-I gene expression in Tg-HuapoA-I mice does not protect from infection with T. b. brucei or T. b. brucei-SRA. FIG. 3A shows A280 profile of KBr-purified lipoproteins separated by size on a Superdex 200 column; fractions 6-7 are void, fractions 10-14 are HDLs as indicated by the distribution of apoA-I, and fraction 15 is albumin. A Western blot of the fractions probed for apoA-I (28 kD) and Tr-apoL-I (˜35 kD) detected with polyclonal anti-apoL-I is shown in the inset of FIG. 3A. FIG. 3B shows western blot with serial dilutions of plasma from HuapoA-I mice expressing Hpr and Tr-apoL-I from a single plasmid (HuapoA-I:Hpr:Tr-apoL-I) and normal human plasma. Hpr, apoL-I, and Tr-apoL-I were detected with polyclonal antibodies. FIG. 3C shows mice expressing the combination of Hpr and apoL-I in a single plasmid (black circle, Hpr:apoL-I; n=3), Tr-apoL-I (cross, n=5), the combination of Hpr and Tr-apoL-I in a single plasmid (gray diamond; Hpr:Tr-apoL-I; n=10) that were infected i.p. with 5,000 T. b. brucei trypanosomes. Control mice were treated with empty vector (gray circle; n=4). The parasitemia was monitored and time of death was recorded. FIG. 3D shows results for mice expressing a combination of Hpr and apoL-I in a single plasmid (black circle, Hpr:apoL-I; n=5) and a combination of Hpr and Tr-apoL-I in a single plasmid (gray diamond; Hpr:Tr-apoL-I; n=5) were infected i.p. with 5,000 T. b. brucei-SRA trypanosomes. Control mice were treated with empty vector (gray circle; n=2). The parasitemia was monitored and the time of death was recorded.

FIGS. 4A-D show mammalian expression constructs. Each construct contains at least one of the following: ubiquitin promoter (arrow), β-globin intron (pink), SV40 poly(A) signal sequence (light blue), β-lactamase, ampicillin resistance gene (grey), bacterial origin of replication (Filled circle) and multiple cloning site (double headed arrow). The following constructs were used FIG. 4A shows the empty plasmid/pRG977, FIG. 4B shows Baboon Hpr gene only (orange)/BH12, FIG. 4C shows Baboon apoL-I only (dark blue)/bL22, and FIG. 4D shows Baboon apoL-I gene (dark blue) and baboon Hpr gene (orange)/bLH14, where each gene is accompanied by a separate promoter, β-globin intron and poly(A) signal sequence.

FIGS. 5A-B show purified baboon TLF that contains Hpr, apoA-I and an apoL-I homologue. FIG. 5A shows baboon TLF proteins (left lane) and total HDL proteins (right lane) separated by reducing SDS PAGE and silver stained (Molecular weights in kDa). FIG. 5B shows ClustalW2 alignment of baboon (upper) (SEQ ID NO: 5) and human (lower) (SEQ ID NO: 2) apoL-I amino acid sequences. Identical residues are shown below the alignment. Underlined sequences of baboon apoL-I were identified by mass-spectrometry. The signal peptide (arrow) and 39 kDa (arrowhead) proteolytic cleavage positions are indicated. Potential N-linked glycosylation sites of baboon apoL-I (N51, N231; above alignment) or human apoL-I (N261; below alignment) are marked with ̂. Every 10th amino acid is denoted with an asterisk.

FIGS. 6A-C show that human serum resistant trypanosomes are killed by mouse serum containing baboon apoL-I and Hpr associated HDLs. Serum or plasma was prepared from transgenic apoA-I (Tg(APOA1)) mice 3 days post injection with plasmid vector or vector containing baboon apoL-I, Hpr or both genes. Lipoproteins were obtained by density gradient centrifugation of plasma. FIG. 6A shows protein absorbance (280 nm) profile of lipoproteins separated by gel filtration. Fraction 7 is the void volume, fractions 10-16 are HDL. FIG. 6B shows western blots of HDL fractions purified from mice expressing apoL-I only, Hpr only, both apoL-I and Hpr. FIG. 6C shows that human serum resistant T. b. brucei (427-SRA) are killed in medium containing serially diluted murine serum. Living trypanosome number after 17 hours is shown as a percentage of the starting number (2.5×10⁵/ml). The data obtained from three independent experiments are presented as mean+/−s.d.

FIG. 7 shows that HDLs containing baboon apoL-I and Hpr kill human serum resistant (infective) trypanosomes in vitro. Tg(APOA1) mice were injected with empty vector or plasmid containing either baboon apoL-I, Hpr or both. Three days post injection, the pooled plasma of 2 mice (1 ml) was subjected to density gradient centrifugation and HDLs were purified from the lipoprotein fraction by gel filtration as shown in FIG. 5. A standard trypanosome killing assay with human infective T. brucei 427-SRA was performed in the presence or absence of 10 mM NH₄Cl. The data is shown as mean+/−s.d. and are representative of three independent experiments.

FIGS. 8A-B show that Baboon apoL-I and Hpr protect against infection with human serum resistant T. b. rhodesiense in Tg(APOA1) mice. Human apoA-I transgenic mice (C57BL/6-Tg(APOAI)1Rub/J) shown in FIG. 8A or murine apoA-I expressing mice (congenic C57BL/6) shown in FIG. 8B were injected with empty vector or a plasmid containing baboon apoL-I or baboon apoL-I+baboon Hpr. After three days mice were infected with 5000 human serum resistant T. b. rhodesiense (Ketri 243 strain) and time of death was recorded.

FIGS. 9A-B show that Baboon apoL-I and Hpr protect mice from infection with T. congolense and human serum resistant trypanosomes. Tg(APOA1) mice were injected with empty vector or a plasmid encoding baboon apoL-I or both baboon apoL-I and Hpr (apoL-I:Hpr). FIG. 9A shows results three days post transfection. Mice were infected i.p. with 5000 human serum resistant T. b. brucei-SRA (427-SRA) and time of death was recorded. apoL-I+Hpr mice were significantly different from vector controls: * P=0.0126 (Log Rank test). FIG. 9B shows results for mice that were infected with 5000 T. congolense (STIB68Q) and blood parasitaemia was monitored (5×10⁴ parasites/ml of blood=Limit of detection). The data are presented as mean+/−s.d. apoL-I:Hpr and apoL-I mice were significantly different from vector controls (days 0-21) and apoL-I mice were significantly different from vector controls during days 0-9: *P=0.0208, **P=0.0015 (two-way analyses of variance).

FIGS. 10A-C show that Baboon apoL-I Lysine 379 and Lysine 380 prevent chimeric apoL-I binding to SRA and confer immunity to human serum resistant trypanosomes. FIG. 10A shows the alignment of the C-terminal 60 amino acids of human apoL-I (SEQ ID NO: 6) with baboon apoL-I (SEQ ID NO: 7) and the human/baboon apoL-I chimera 1 (SEQ ID NO: 8), human/baboon apoL-I chimera 2 (SEQ ID NO: 9), human/baboon apoL-I chimera 3 (SEQ ID NO: 10), human/baboon apoL-I chimera 4 (SEQ ID NO: 11), and human/baboon apoL-I chimera 5 (SEQ ID NO: 12). Amino acid characteristics are represented as follows, Italics: hydrophobic residues (A, F, G, I, L, P, V, W, Y); red: basic residues (K, R, H); green: acidic residues (D, E); blue: hydrophilic residues (N, Q, S, T) and cysteine in plain text. Asterisks denote amino acid differences. The PHD secondary structure prediction for human apoL-I is shown (E=extended, C=coil, H=helix). Swiss Webster mice were injected with empty plasmid (vector) or plasmids encoding human apoL-I or chimeric apoL-I in FIG. 10B. After 3 days, mice were infected with 5000 T. b. brucei-SRA (427-SRA). Chimera 4 survival was significantly different from vector controls, *P<0.0001 and chimeras 1-3, *P=0.0183 (log rank test). Survival of chimeras 1-3 was significantly different from vector controls: **P=0.0005 (log rank test). FIG. 10C shows data relating to normal human serum (NHS) or the serum from mice transfected with empty vector, human apoL-I or chimeric apoL-I which was incubated with His-tagged SRA at pH 5.8 and SRA complexes were bound to Ni-NTA agarose. apoL-I was detected in the bound fraction (bound) or in an equivalent dilution of whole serum (load) by western blotting with anti-human apoL-I polyclonal IgG.

FIG. 11 shows that Baboon TLF does not bind to SRA. Purified human or baboon HDL was incubated with His-tagged SRA at pH 5.8 and SRA complexes were bound to Ni-NTA agarose. Hpr was detected in whole HDL (load) or in the bound fraction in the presence or absence of SRA by western blotting using anti-human Haptoglobin polyclonal IgG.

### DETAILED DESCRIPTION OF THE INVENTION

A first aspect of the present invention is directed to a Trypanosome-resistant, non-human transgenic animal whose somatic and germ cells comprise a first nucleic acid transgene which encodes an apolipoprotein L-I polypeptide (apoL-I). The apoL-I protein has the amino acid sequence selected from the group consisting of SEQ ID NO: 1, SEQ ID NO: 3, SEQ ID NO: 4, and SEQ ID NO: 5. The first nucleic acid transgene is operatively associated with at least one expression regulatory sequence. The apolipoprotein L-I polypeptide has the following amino acid sequence (SEQ ID NO: 1):

| | |
|---|---|
| 1 | MEGAALLRLF VLCIWTSGLF LGVGVRAEEA TARVQQNHPG GTDTGDPQES |
| 51 | NVSLEDYLNY FQKNVSPQEM LLLLSDHKAW ERVVATAELP RDEADELYKA |
| 101 | LNKLIRHMVM KDKNWLEEVQ QHRKRFLEEF PRLERELQDK IRRLCDLAGQ |
| 151 | VQKVHKGATI ANAFSSTLGV ASGVLTFLGL GLAPFTAGSS LVLLEPVTGL |
| 201 | GIAAALTGIT SGSVEYAKKR WAQAEAHELV NKSLDTVEEM NEFLYHNIPN |
| 251 | FISLRVNLVK FTEDTGKAIR AIRQARANPH SVSHVPASLH RVTEPVSATS |
| 301 | VEERARVVEM ERVAESRTTE VIRGAKIVDK VFEGALFVLD VVGLVCQLKH |
| 351 | LHEGAKSXTA XXLKKVAQXL XXXLZILZXX YXTLRQXP |

where X can be E, R, H, or K;
where Z can be N, T, K, or R.

The human apolipoprotein L-I (SEQ ID NO: 2) has the following amino acid sequence:

| | |
|---|---|
| 1 | MEGAALLRVS VLCIWMSALF LGVGVRAEEA GARVQQNVPS GTDTGDPQSK |
| 51 | PLGDWAAGTM DPESSIFIED AIKYFKEKVS TQNLLLLLTD NEAWNGFVAA |
| 101 | AELPRNEADE LRKALDNLAR QMIMKDKNWH DKGQQYRNWF LKEFPRLKSE |
| 151 | LEDNIRRLRA LADGVQKVHK GTTIANVVSG SLSISSGILT LVGMGLAPFT |
| 201 | EGGSLVLLEP GMELGITAAL TGITSSTMDY GKKWWTQAQA HDLVIKSLDK |
| 251 | LKEVREFLGE NISNFLSLAG NTYQLTRGIG KDIRALRRAR ANLQSVPHAS |
| 301 | ASRPRVTEPI SAESGEQVER VNEPSILEMS RGVKLTDVAP VSFFLVLDVV |
| 351 | YLVYESKHLH EGAKSETAEE LKKVAQELEE KLNILNNNYK ILQADQEL |

The SEQ ID NO: 3 is a mutated sequence of human apoliprotein L-I (SEQ ID NO: 2) with N387K and N388K mutations (both mutated positions are underlined) and the following amino acid sequence:

| | |
|---|---|
| 1 | MEGAALLRVS VLCIWMSALF LGVGVRAEEA GARVQQNVPS GTDTGDPQSK |
| 51 | PLGDWAAGTM DPESSIFIED AIKYFKEKVS TQNLLLLLTD NEAWNGFVAA |
| 101 | AELPRNEADE LRKALDNLAR QMIMKDKNWH DKGQQYRNWF LKEFPRLKSE |
| 151 | LEDNIRRLRA LADGVQKVHK GTTIANVVSG SLSISSGILT LVGMGLAPFT |
| 201 | EGGSLVLLEP GMELGITAAL TGITSSTMDY GKKWWTQAQA HDLVIKSLDK |
| 251 | LKEVREFLGE NISNFLSLAG NTYQLTRGIG KDIRALRRAR ANLQSVPHAS |
| 301 | ASRPRVTEPI SAESGEQVER VNEPSILEMS RGVKLTDVAP VSFFLVLDVV |
| 351 | YLVYESKHLH EGAKSETAEE LKKVAQELEE KLNILNKKYK ILQADQEL |

The SEQ ID NO: 4 has the following amino acid sequence:

| | |
|---|---|
| 1 | MEGAALLRVS VLCIWMSALF LGVGVRAEEA GARVQQNVPS GTDTGDPQSK |
| 51 | PLGDWAAGTM DPESSIFIED AIKYFKEKVS TQNLLLLLTD NEAWNGFVAA |
| 101 | AELPRNEADE LRKALDNLAR QMIMKDKNWH DKGQQYRNWF LKEFPRLKSE |
| 151 | LEDNIRRLRA LADGVQKVHK GTTIANVVSG SLSISSGILT LVGMGLAPFT |
| 201 | EGGSLVLLEP GMELGITAAL TGITSSTMDY GKKWWTQAQA HDLVIKSLDK |
| 251 | LKEVREFLGE NISNFLSLAG NTYQLTRGIG KDIRALRRAR ANLQSVPHAS |
| 301 | ASRPRVTEPI SAESGEQVER VNEPSILEMS RGVKLTDVAP VSFFLVLDVV |
| 351 | YLVYESKHLH EGAKSETAEE LKKVAQELEE KLNILNXXYX TLRQXP |

where any amino acids in the residues from A339 to N386 (underlined) could be replaced, respectively, with the corresponding amino acids from SEQ ID NO: 13 which has the following amino acid sequence:

| | |
|---|---|
| 339 | VFEGALFVLD VVGLVCQLKH LHEGAKSXTA XXLKKVAQXL |
| | XXXLZILZ |

where X can be E, R, H, or K;
where Z can be N, T, K, or R.

In a preferred embodiment of this aspect of the present invention, the transgenic animal has the apolipoprotein L-I protein from baboon (Papio hamadryas). The amino acid sequence of baboon apolipoprotein L-I (SEQ ID NO: 5) is as follows:

| | |
|---|---|
| 1 | MEGAALLRLF VLCIWTSGLF LGVGVRAEEA TARVQQNHPG GTDTGDPQES |
| 51 | NVSLEDYLNY FQKNVSPQEM LLLLSDHKAW ERVVATAELP RDEADELYKA |
| 101 | LNKLIRHMVM KDKNWLEEVQ QHRKRFLEEF PRLERELQDK IRRLCDLAGQ |
| 151 | VQKVHKGATI ANAFSSTLGV ASGVLTFLGL GLAPFTAGSS LVLLEPVTGL |
| 201 | GIAAALTGIT SGSVEYAKKR WAQAEAHELV NKSLDTVEEM NEFLYHNIPN |
| 251 | FISLRVNLVK FTEDTGKAIR AIRQARANPH SVSHVPASLH RVTEPVSATS |
| 301 | VEERARVVEM ERVAESRTTE VIRGAKIVDK VFEGALFVLD VVGLVCQLKH |
| 351 | LHEGAKSKTA EELKKVAQEL EKKLNILNKK YETLRQEP |

By “transgene” is meant any nucleotide or DNA sequence that is integrated into one or more chromosomes of a host cell by human intervention, such as by the methods of the present invention. In one embodiment, the transgene comprises a “gene of interest.” A “gene of interest” is a nucleic acid sequence that encodes a protein or other molecule that is desirable for integration and/or expression in a host cell. In this embodiment, the gene of interest is generally operatively linked to other sequences that are useful for obtaining the desired expression of the gene of interest, such as transcriptional regulatory sequences.

The term “transgenic” is used herein to describe the property of harboring a transgene. For instance, a “transgenic organism” is any animal, including mammals, fish, birds, and amphibians, in which one or more of the cells of the animal contain nucleic acid introduced by way of human intervention, such as by the methods described herein. In the typical transgenic animal, the transgene causes the cell to express or over express a recombinant protein.

Generation of the nucleic acid construct can be accomplished using any suitable genetic engineering techniques well known in the art, including, without limitation, the standard techniques of restriction endonuclease digestion, ligation, transformation, plasmid purification, and DNA sequencing, for example as described in Sambrook et al. Molecular Cloning: A Laboratory Manual (1989), which is hereby incorporated by reference in its entirety.

The transgene can be carried in host cells. Host cells include plant, bacterial, or animal cells, which may be used to maintain or propagate the transgene. Host cells also encompasses mammalian cells which have been transformed with the transgene. The invention also encompasses a cell line, clone, or tumor derived from a transgenic mammal. In particular, the invention includes an embryonic stem cells which contain the transgenic substrate.

Apoliprotein L-I (apoL-I) is a protein found in trypanolytic high-density lipoprotein particles, known as Trypanosome lytic factor (TLF). The full length amino acid sequences of human apoL-I are well known in the art (see GenBank Accession No. AAI43040, which is hereby incorporated by reference in its entirety). As shown herein, the apoL-I protein plays an important role in imparting resistance against trypanosomes.

In one embodiment, the transgenic animal has a stable plasma level of apolipoprotein L-I polypeptide throughout its sexual maturity.

The gene of interest in the present invention is preferably in a functional relationship with other genetic elements, for example transcription regulatory sequences such as promoters and/or enhancers, to regulate expression of the gene of interest in a particular manner once the transgene is incorporated into the host genome. In certain embodiments, the useful transcriptional regulatory sequences are those that are highly regulated with respect to activity, both temporally and spatially.

The expression regulatory sequence is preferably selected based on the desired expression pattern of the gene of interest and the specific properties of known promoters/enhancers. Thus, the internal promoter may be a constitutive promoter. Alternatively, the promoter may be a tissue specific promoter. In addition, promoters may be selected to allow for inducible expression of the transgene. A number of systems for inducible expression using such a promoter are known in the art, including the tetracycline responsive system and the lac operator-repressor system. It is also contemplated that a combination of promoters may be used to obtain the desired expression of the gene of interest. The skilled artisan will be able to select a promoter based on the desired expression pattern of the gene in the resulting transgenic animal.

The expression regulatory sequence controlling the expression of proteins of the present invention can be a promoter sequence selected from the group consisting of a viral promoter, an apoA-I promoter, an apoL-I promoter, a hepatic enhancer of apoE, an intestinal enhancer of apoCIII, a ubiquitous promoter, a filament promoter, an MDR promoter, a CFTR promoter, factor promoter, a tissue-specific promoter, a promoter which is preferentially activated in dividing cells, a promoter which responds to a stimulus, a tetracycline-regulated transcriptional modulator, and a metallothionein promoter.

In another embodiment of the present invention, the Trypanosome-resistant non-human transgenic animal whose somatic and germ cells comprise a nucleic acid transgene which encodes an apolipoprotein L-I polypeptide (apoL-I) further comprise a second nucleic acid transgene encoding a haptoglobin related protein (Hpr). The Hpr encoding nucleic acid transgene is operatively associated with at least one expression regulatory sequence. Haptoglobin related protein (Hpr) is another protein found in trypanolytic high-density lipoprotein particles, known as Trypanosome lytic factor (TLF). The full length amino acid sequences of human Hpr are well known in the art (see GenBank Accession No. NP_{—}066275, which is hereby incorporated by reference in its entirety). As shown herein, the Hpr protein plays an important role in imparting resistance against trypanosomes.

The human Hpr polypeptide has the following amino acid sequence (SEQ ID NO: 14):

| | |
|---|---|
| 1 | MSDLGAVISL LLWGRQLFAL YSGNDVTDIS DDRFPKPPEI ANGYVEHLFR |
| 51 | YQCKNYYRLR TEGDGVYTLN DKKQWINKAV GDKLPECEAV CGKPKNPANP |
| 101 | VQRILGGHLD AKGSFPWQAK MVSHHNLTTG ATLINEQWLL TTAKNLFLNH |
| 151 | SENATAKDIA PTLTLYVGKK QLVEIEKVVL HPNYHQVDIG LIKLKQKVLV |
| 201 | NERVMPICLP SKNYAEVGRV GYVSGWGQSD NFKLTDHLKY VMLPVADQYD |
| 251 | CITHYEGSTC PKWKAPKSPV GVQPILNEHT FCVGMSKYQE DTCYGDAGSA |
| 301 | FAVHDLEEDT WYAAGILSFD KSCAVAEYGV YVKVTSIQHW VQKTIAEN |

The human Hpr polypeptide is encoded by the following nucleic acid sequence (SEQ ID NO: 15):

| | |
|---|---|
| 1 | actgctcttc cagaggcaag accaaccaag atgagtgacc tgggagctgt catttccctc |
| 61 | ctgctctggg gacgacagct ttttgcactg tactcaggca atgatgtcac ggatatttca |
| 121 | gatgaccgct tcccgaagcc ccctgagatt gcaaatggct atgtggagca cttgtttcgc |
| 181 | taccagtgta agaactacta cagactgcgc acagaaggag atggagtata caccttaaat |
| 241 | gataagaagc agtggataaa taaggctgtt ggagataaac ttcctgaatg tgaagcagta |
| 301 | tgtgggaagc ccaagaatcc ggcaaaccca gtgcagcgga tcctgggtgg acacctggat |
| 361 | gccaaaggca gctttccctg gcaggctaag atggtttccc accataatct caccacaggg |
| 421 | gccacgctga tcaatgaaca atggctgctg accacggcta aaaatctctt cctgaaccat |
| 481 | tcagaaaatg caacagcgaa agacattgcc cctactttaa cactctatgt ggggaaaaag |
| 541 | cagcttgtag agattgagaa ggtggttcta caccctaact accaccaggt agatattggg |
| 601 | ctcatcaaac tcaaacagaa ggtgcttgtt aatgagagag tgatgcccat ctgcctacct |
| 661 | tcaaagaatt atgcagaagt agggcgtgtg ggttacgtgt ctggctgggg acaaagtgac |
| 721 | aactttaaac ttactgacca tctgaagtat gtcatgctgc ctgtggctga ccaatacgat |
| 781 | tgcataacgc attatgaagg cagcacatgc cccaaatgga aggcaccgaa gagccctgta |
| 841 | ggggtgcagc ccatactgaa cgaacacacc ttctgtgtcg gcatgtctaa gtaccaggaa |
| 901 | gacacctgct atggcgatgc gggcagtgcc tttgccgttc acgacctgga ggaggacacc |
| 961 | tggtacgcgg ctgggatcct aagctttgat aagagctgtg ctgtggctga gtatggtgtg |
| 1021 | tatgtgaagg tgacttccat ccagcactgg gttcagaaga ccatagctga gaactaatgc |
| 1081 | aaggctggcc ggaagccctt gcctgaaagc aagatttcag cctggaagag ggcaaagtgg |
| 1141 | acgggagtgg acaggagtgg atgcgataag atgtggtttg aagctgatgg gtgccagccc |
| 1201 | tgcattgctg agtcaatcaa taaagagctt tcttttgacc caaaa |

The baboon Hpr polypeptide has the following amino acid sequence (SEQ ID NO: 16) (see GenBank Accession No. AY552414.2, which is hereby incorporated by reference in its entirety):

| | |
|---|---|
| 1 | MSDLGAVISL LLWGRQLFAL YSGNDVTDIP DDSFLKPPMI ANGYVEHLVR |
| 51 | YQCKSYYRLR TEGDGVYTLN NEKQWTNKAV GDKLPECEAV CGKPKNPADA |
| 101 | VQRILGGHLD AKGSFPWQAK MVSRHNLTTG ATLINEQWLL TTAKNLFLNH |
| 151 | SENATAKDIA PTLTLYVGKK QLVEIEKVVL HPNYSQVDIG LIKLKQKVPV |
| 201 | NERVMPICLP SKDYAEVGRV GYVSGWGRNA NFNFTDHLKY VMLPVADQYD |
| 251 | CIKHYEGSKC PKRKAPKNPV GVQPILNEHT FCAGMSKYQE DTCYGDAGTA |
| 301 | FAVHDMEEDT WYAAGILSFD KSCGVAKYGV YVKATSIQDW VQKTIAEN |

The baboon Hpr polypeptide is encoded by the following nucleic acid sequence (SEQ ID NO: 17):

| | |
|---|---|
| 1 | atgagtgacc tgggagctgt catttccctc ctgctctggg gacgacagct ttttgcattg |
| 61 | tactcaggca atgatgtcac ggatatccca gatgacagct tcctgaagcc ccctatgatc |
| 121 | gcaaatggct acgtggagca cttggttcgc taccagtgta agagctacta caggctgcgc |
| 181 | acagaaggag atggagtgta caccttaaac aatgagaagc agtggacaaa taaggctgtt |
| 241 | ggagataaac ttcctgaatg tgaagcagtg tgtgggaagc ccaagaatcc ggcagacgca |
| 301 | gtgcagcgga tcctgggtgg acacctggat gccaaaggca gctttccctg gcaggctaag |
| 361 | atggtttccc gccataatct caccacaggg gccacgctga tcaatgaaca atggctgctg |
| 421 | accacggcta aaaatctctt cctgaaccat tcagaaaatg caacagcgaa agacattgcc |
| 481 | cctactttaa cactctatgt ggggaaaaag cagcttgtag agattgagaa ggtggttcta |
| 541 | caccctaact actcccaggt agatattggg ctcatcaaac tcaaacagaa ggtgcctgtt |
| 601 | aatgagagag tgatgcccat ctgcctaccc tcaaaggatt atgcagaagt agggcgtgtg |
| 661 | ggttacgtgt ctggctgggg gcgaaatgcc aattttaact ttactgacca tctgaagtat |
| 721 | gtcatgctgc ctgtggctga ccaatacgat tgcataaagc actatgaagg cagcaaatgc |
| 781 | cccaaaagga aggcaccgaa gaaccctgta ggggtgcagc ccatactgaa tgaacacacc |
| 841 | ttctgtgccg gcatgtctaa gtaccaggaa gacacctgct atggcgatgc aggcactgcc |
| 901 | tttgccgttc acgacatgga ggaggacacc tggtatgcag ctgggatcct aagctttgat |
| 961 | aagagctgtg gtgtggctaa gtatggtgtg tatgtgaagg cgacttccat ccaggactgg |
| 1021 | gttcagaaga ccatagctga gaactaa |

The transgenic animal of the present invention can be, without limitation, a mammal. In a preferred embodiment, the transgenic animal is selected from the group consisting of mouse, rat, pig, sheep, bovine animals, camel, and horse. The present invention is specially directed towards animals such as livestock and animals of burden that are prone to diseases caused by Trypanosomes.

The transgenic animal of the present invention is such that the first nucleic acid transgene encoding apoL-I polypeptide and second nucleic acid transgene encoding haptoglobin binding protein can be linked by nucleic acid linkers well known in the art such that both of the two protein are expressed in the animal. For example, a particularly useful sequence can have both baboon apoL-I and baboon Hpr linked together.

The “Trypanosome-resistant, non-human transgenic animals” of the present invention can be produced by introducing “transgenes” into the germline of the non-human animal. The method provides for transgenic animals whose somatic and germ cells contain transgene encoding for apolipoprotein L-I protein and/or other proteins of the present invention. The transgenic animals can be produced by techniques well known in the art. Any method can be used which provides for stable, inheritable, expressible incorporation of the transgene within the nuclear DNA of an animal.

Another aspect of the present invention is related to a method of producing a Trypanosome-resistant, non-human transgenic animal. This method comprises providing a first nucleic acid construct. This first nucleic acid construct has a first nucleic acid molecule which encodes for apolipoprotein L-I polypeptide having the amino acid sequence of SEQ ID NO: 1, SEQ ID NO: 3, SEQ ID NO: 4, and SEQ ID NO: 5. The first nucleic acid molecule is operably associated with at least one expression regulatory sequence. The method further involves introducing the first nucleic acid construct into a zygote of a non human animal and then transplanting the zygote into a pseudopregnant non-human female animal. This transplanted zygote is allowed to develop to term and at least one transgenic offspring containing the first nucleic acid molecule is subsequently identified.

A nucleic acid molecule that encodes for haptoglobin binding protein (Hpr) may be co-introduced into the zygote of a non-human animal. An Hpr encoding nucleic acid molecule can be co-introduced as a part of the first nucleic acid construct or as a separate nucleic acid construct.

In one embodiment of this aspect of the present invention, the first nucleic acid construct further comprises a second nucleic acid molecule that encodes a haptoglobin binding protein (Hpr). The Hpr encoding nucleic acid sequence is operatively associated with at least one expression regulatory sequence.

In carrying out this aspect of the present invention, a second nucleic acid construct comprising a nucleic acid molecule which encodes a haptoglobin binding protein (Hpr) is provided. The second nucleic acid molecule is operatively associated with at least one expression regulatory sequence and introduced into a zygote of the non-human animal into which the first nucleic acid construct is already introduced. The zygote is then transplanted into a pseudopregnant non-human female animal.

The particular forms of the apolipoprotein L-I polypeptide and Hpr, as described above, can also be used in this aspect of the present invention.

The zygote is the best target for introduction of transgene by micro-injection. The use of zygotes as a target for gene transfer has a major advantage in that in most cases the injected DNA will be incorporated into the host gene before the first cleavage (Brinster, et al., “Factors Affecting the Efficiency of Introducing Foreign DNA into Mice by Microinjecting Eggs,” Proc Nat'l Acad Sci USA 82:4438-4442 (1985), which is hereby incorporated by reference in its entirety). As a consequence, all cells of the transgenic non-human animal will carry the incorporated transgene. This will in general also be reflected in the efficient transmission of the transgene to offspring of the founder since 50% of the germ cells will harbor the transgene.

One means available for producing a transgenic animal (e.g., a mouse) is as follows: Female mice are mated, and the resulting fertilized eggs are dissected out of their oviducts. The eggs are stored in an appropriate medium such as M2 medium (Hogan B. et al., “Manipulating the Mouse Embryo, A Laboratory Manual,” Cold Spring Harbor Laboratory (1986), which is hereby incorporated by reference in its entirety). A DNA or cDNA encoding gene, minigene or a recombinatorial substrate is purified from a vector (such as plasmids) by methods well known in the art. Inducible promoters may be fused with the coding region of the DNA to provide an experimental means to regulate expression of the transgene. Alternatively or in addition, tissue specific regulatory elements may be fused with the coding region to permit tissue-specific expression of the transgene. The DNA, in an appropriately buffered solution, is put into a microinjection needle (which may be made from capillary tubing using a pipet puller), and the egg to be injected is put in a depression slide. The needle is inserted into the pronucleus of the egg, and the DNA solution is injected. The injected egg is then transferred into the oviduct of a pseudopregnant mouse (i.e., a mouse stimulated by the appropriate hormones to maintain pregnancy but which is not actually pregnant), where it proceeds to the uterus, implants, and develops to term. This pronuclear DNA microinjection is a classic method for gene transfer and is described by Brem, Transgenic Animals pp. 745-832 (1993); and Hammer et al., “Production of Transgenic Rabbits, Sheep and Pigs by Microinjection,” Nature 315:680-683 (1985), which are hereby incorporated by reference in their entirety. The basic method for DNA microinjection as used in mice is described in Rulicke et al., “Germline Transformation of Mammals by Pronuclear Microinjection,” Experimental Physiology 85:589-601 (2000), which is hereby incorporated by reference in its entirety. However, some species specific modifications are necessary in utilizing this method. For example, in the recovery of embryos, the microinjection process and the transfer of injected embryos to the recipients may require such modification (Brem, Transgenic Animals pp. 745-832 (1993), which is hereby incorporated by reference in its entirety). DNA microinjection is not the only method for inserting DNA into the egg cell, and is used here only for exemplary purposes.

Another aspect of this invention is directed to a method for producing a Trypanosome-resistant, non-human transgenic animal using an embryo. A first nucleic acid construct is provided which is comprised of a first nucleic acid molecule encoding an apolipoprotein L-I polypeptide having the amino acid sequence selected from the group consisting of SEQ ID NO: 1, SEQ ID NO: 3, SEQ ID NO: 4, and SEQ ID NO: 5. This first nucleic acid molecule is operably associated with at least one expression regulatory sequence. The first nucleic acid construct is then introduced into an embryo of a non-human animal and the embryo is transplanted into a pseudopregnant non-human female animal. The embryo is allowed to develop to term, and a transgenic offspring containing the first nucleic acid molecule is subsequently identified.

In one embodiment of this aspect of the present invention, the first nucleic acid construct further comprises a second nucleic acid molecule that encodes a haptoglobin binding protein (Hpr). The Hpr encoding nucleic acid sequence is operatively associated with at least one expression regulatory sequence.

In another embodiment of this aspect of the present invention, the method further comprises providing a second nucleic acid construct comprising a nucleic acid molecule which encodes a haptoglobin binding protein (Hpr). The second nucleic acid molecule is operatively associated with at least one expression regulatory sequence. The method further involves introducing the second nucleic acid construct into the embryo of the non-human animal into which the first nucleic acid construct is already introduced. The embryo is then transplanted into the pseudopregnant non-human female animal.

The introducing step of the nucleic acid constructs may also be carried out by several methods well known in the art, which include, but are not limited to, a) microinjection of genes into the pronuclei of fertilized ova; b) DNA transfer by retroviruses; c) injection of embryonic stem cells and/or embryonic germ cells, previously exposed to foreign DNA, into the cavity of blastocysts; d) sperm mediated exogenous DNA transfer during in vitro fertilization; e) liposome-mediated DNA transfer into cells and embryos; f) electroporation of DNA into sperms, ova or embryos; g) biolistics; and h) nuclear transfer with somatic or embryonic cells, which are described in Wheeler et al., “Transgenic Technology and Applications in Swine,” Theriogenology 56:1345-1370 (2001); Wolf et al., “Transgenic Technology in Farm Animals—Progress and Perspectives,” Exp Physiol 85.6:615-625 (2000), which are hereby incorporated by reference in their entirety.

The particular forms of the apolipoprotein L-I polypeptide and Hpr, as described above, can also be used in this aspect of the present invention.

In one embodiment of the present invention, the nucleic acid constructs can be introduced into the embryo by infecting the embryo with a virus containing the nucleic acid construct. A recombinant retrovirus may be used to deliver a transgene of interest to a cell, preferably an oocyte or an embryonic cell, more preferably a one-cell embryo. The transgene and any associated genetic elements, are thus integrated into the genome of the host cell as a provirus. The cell may then be allowed to develop into a transgenic animal. The developing non-human embryo can be cultured in vitro to the blastocyst stage. During this time, the blastomeres can be targets for retroviral infection (Jaenich, “Germ Line Integration and Mendelian Transmission of the Exogenous Moloney Leukemia Virus,” Proc Nat'l Acad Sci USA 73:1260-1264 (1976), which is hereby incorporated by reference in its entirety). Efficient infection of the blastomeres is obtained by enzymatic treatment to remove the zona pellucida (Hogan, et al., Manipulating the Mouse Embryo (1986), which is hereby incorporated by reference in its entirety). The viral vector system used to introduce the transgene is typically a replication-defective retrovirus carrying the transgene (Jahner et al., “Insertion of the Bacterial gpt Gene into the Germ Line of Mice by Retroviral Infection,” Proc Nat'l Acad Sci USA 82:6927-6931 (1985); Van der Putten et al., “Efficient Insertion of Genes into the Mouse Germ Line via Retroviral Vectors,” Proc Nat'l Acad Sci USA 82:6148-6152 (1985), which are hereby incorporated by reference in their entirety). Transfection is easily and efficiently obtained by culturing the blastomeres on a monolayer of virus-producing cells (Van der Putten et al., “Efficient Insertion of Genes into the Mouse Germ Line via Retroviral Vectors,” Proc Nat'l Acad Sci USA 82:6148-6152 (1985); Stewart et al., “Expression of Retroviral Vectors in Transgenic Mice Obtained by Embryo Infection,” EMBO J. 6:383-388 (1987), which are hereby incorporated by reference in their entirety). Alternatively, infection can be performed at a later stage. Virus or virus-producing cells can be injected into the blastocoele (Jahner et al., “De novo Methylation and Expression of Retroviral Genomes during Mouse Embryogenesis,” Nature 298:623-628 (1982), which is hereby incorporated by reference in its entirety).

In another embodiment of the invention, the nucleic acid constructs of the present invention can be introduced into the embryo by introducing an embryonic stem cell containing the nucleic acid construct into the embryo. Embryonic stem (“ES”) cells at various developmental stages can be used to introduce transgenes. Different methods are used depending on the stage of development of the embryonal cell. ES cells may be obtained from pre-implantation embryos cultured in vitro and fused with embryos (Evans et al., “Establishment in culture of Pluripotential Cells from Mouse Embryos,” Nature 292:154-156 (1981); Gossler et al., “Transgenesis by Means of Blastocyst-derived Embryonic Stem Cell Lines,” Proc Nat'l Acad Sci USA 83:9065-9069 (1986), which are hereby incorporated by reference in their entirety). Transgenes can be efficiently introduced into the ES cells by DNA transfection or by retrovirus mediated transduction. Such transformed ES cells can thereafter be combined with blastocysts from a nonhuman animal. The ES cells thereafter colonize the embryo and contribute to the germ line of the resulting chimeric animal (Jaenisch, “Transgenic Animals,” Science 240:1468-1474 (1988), which is hereby incorporated by reference in its entirety). For example, one method that can be used is laser-assisted injection of either inbred or hybrid ES cells into eight cell-stage embryos (Poueymirou et al., “F0 Generation Mice Fully Derived from Gene-targeted Embryonic Stem Cells Allowing Immediate Phenotypic Analyses,” Nat Biotech 25:91-99 (2007), which is hereby incorporated by reference in its entirety). This method efficiently yields F0 generation mice that are fully ES cell-derived and healthy, exhibit 100% germline transmission and allow immediate phenotypic analysis, greatly accelerating gene function assignment.

Animals carrying the transgene may be selected by identifying animals which carry a reporter gene contained in the transgenic construct. Such identifying may be carried out by screening for a protein expressed by the gene. For example, by using antibodies specific to the protein which is expressed. The antibodies may also be chemically or radioactively tagged to facilitate detection. The identifying step may also be carried out by screening for a phenotype conferred by the gene. An example would be screening for drug resistance or fluorescence. Such identification may be further carried out by directly screening for the gene, gene product or an RNA molecule made by the gene using nucleic acid hybridization techniques. For example, but not limited to, diagnostic dip stick test could be used for presence of apoL-I in blood of an animal, where a dip stick coated with an antibody and a colorimetric reporter is used.

In one embodiment of the present invention the offspring having the first nucleic acid molecule encoding for apolipoprotein L-I polypeptide may be bred further to form a non-human transgenic animal.

Another aspect of the present invention is directed to a transgenic egg comprising a first nucleic acid transgene which encodes an apolipoprotein L-I polypeptide having the amino acid sequence selected from the group consisting of SEQ ID NO: 1, SEQ ID NO: 3, SEQ ID NO: 4, and SEQ ID NO: 5. This apolipoprotein L-I encoding nucleic acid transgene is operatively associated with at least one expression regulatory sequence. The transgenic egg can further comprise a second nucleic acid transgene sequence which encodes a haptoglobin binding protein (Hpr). This haptoglobin binding protein has the amino acid sequence described above and is operatively associated with at least one expression regulatory sequence.

The Hpr generally as well as the particular forms of the apolipoprotein L-I polypeptide and Hpr, as described above, can also be used in this aspect of the present invention.

Another aspect of the present invention is directed to a transgenic sperm comprising a first nucleic acid transgene which encodes an apolipoprotein L-I polypeptide having the amino acid sequence selected from the group consisting of SEQ ID NO: 1, SEQ ID NO: 3, SEQ ID NO: 4, and SEQ ID NO: 5 and operatively associated with at least one expression regulatory sequence. The transgenic sperm further comprises a second nucleic acid transgene which encodes a haptoglobin binding protein (Hpr). Hpr has one of the amino acid sequences described above and is operatively associated with at least one expression regulatory sequence.

The Hpr generally as well as the particular forms of the apolipoprotein L-I polypeptide and Hpr, as described above, can also be used in this aspect of the present invention.

Another aspect of the present invention is directed to a method of animal husbandry where Trypanosome-resistant, non-human transgenic animals are bred and raised. These non-human transgenic animals have somatic and germ cells comprising a nucleic acid transgene which encodes for an apolipoprotein L-I polypeptide (apoL-I). Animal breeding is the selective mating of animals to increase the possibility of obtaining desired phenotype. This process is well known in the art and has been performed with most domesticated and agricultural animals. Modern procedures are beginning to supplant traditional breeding methods, which focus on selectively combining and isolating livestock strains. In general, the most effective strategy for isolating traits is by selective inbreeding; however, different strains can sometimes be crossed to take advantage of hybrid vigor and to forestall the negative results of inbreeding.

The Hpr generally as well as the particular forms of the apolipoprotein L-I polypeptide and Hpr, as described above, can also be used in this aspect of the present invention.

The modern techniques involve a wide variety of laboratory methods, for example, but not limited to, artificial insemination, modification of embryos, sex selection, genetic engineering. In the present invention, the breeding, raising and maintaining of transgenic animals is required. The breeding can be performed through controlled mating and reproduction of transgenic animals which were selected and mated based on the inclusion of the desired gene and/or a desired phenotype. Genotype refers to the information contained in an animal's DNA, or genetic material. An animal's phenotype is the physical expression of its genotype. In the present invention, the desired phenotype is resistance to Trypanosoma spp. Additionally, an optimal environment for raising the transgenic animal to maturity has to be established (i.e., the proper nutrition and care need to be determined). Good husbandry and agricultural practice can contribute to virological and microbiological safety as well as disease resistant transgenic animals.

In this aspect of the present invention, the transgenic animals, which are fully described above, are raised in an environment where the transgenic animals are susceptible to trypanosome infection. The conditions under which the animals are bred and maintained could be such that they are exposed to Trypanosoma spp. The site where animals are kept should be free of other diseases that are likely to affect the production animal species prior to or during breeding. Potential sources of unwanted infection may include foodstuff, animal handlers, and veterinary surgeons, and the environment especially if the animals are kept outside. The health of the animals should be regularly monitored.

Trypanosomiasis or trypanosomosis is the name of several diseases in vertebrates caused by parasitic protozoan trypanosomes of the genus Trypanosoma. Trypanosomes are a group of kinetoplastid protozoa distinguished by having only a single flagellum. All members are exclusively parasitic. Various Trypanosoma spp. can cause diseases in the animals. Trypanosoma brucei gambiense and Trypanosoma brucei rhodesiense are causative agents of sleeping sickness in humans. Trypanosoma brucei brucei, Trypanosoma congolense and Trypanosoma vivax cause nagana in cattle. Trypanosoma evansi causes surra in camels and Trypanosoma equiperdum causes dura in horses. Besides, those listed above, the trypanosome present during raising and breeding the transgenic animal can be Trypanosoma suis, Trypanosoma evansi, Trypanosoma equinum, Trypanosoma rangeli, and Trypanosoma simiae.

The present invention is illustrated, but not limited, by the following examples.

### Examples

### Example 1

### Cloning and Expression of Apolipoprotein L-I and Haptoglobin-Related Protein in Individual Plasmids and Together in One Plasmid

pRG977 plasmid was obtained from Regeneron Pharmaceuticals, Inc. Human haptoglobin-related protein encoding the full-length signal peptide (GenBank Accession No. NM_{—}020995) was cloned into pRG977 plasmid. A Kozac sequence CCACC was introduced by site-directed mutagenesis (Stratagene; P1).

A TopoTA-PCR product of apolipoprotein L-I from human liver (GenBank Accession No. O14791) was cloned into pRG977 plasmid (P2). The DNA sequence encoding the full-length signal peptide of apoL-I and a Kozac sequence CCACC was introduced by PCR amplification with Pfu Ultra (Stratagene) and cloned into pRG977 (P3).

A dual construct containing apoL-I and Hpr (each with their own promotor, and poly[A] tail) was cloned into pRG977 (P5). Tr-apoL-I and Hpr:Tr-apoL-I was obtained by site-directed mutagenesis of full-length apoL-I, P3, or the dual construct P5 by introducing a stop codon at position 342. All plasmids were sequenced before use.

### Example 2

### Transfection of Mice

Expression of human Hpr and ApoL-I in the plasma of mice was achieved by performing hydrodynamics-based transfection (Kobayashi, et al., “Hydrodynamics-based Procedure Involves Transient Hyperpermeability in the Hepatic Cellular Membrane: Implication of a Nonspecific Process in Efficient Intracellular Gene Delivery,” J. Gene Med. 6:584-592 (2004), which is hereby incorporated by reference in its entirety). Male Swiss Webster mice (20 g) were injected i.v., in less than 10 s, with 2 ml of sterile 0.9% NaCl solution containing 50-100 μg of plasmids. For expression of apoL-I, Hpr, or both in HDL particles containing human apoA-I, high-volume injections were performed in C57BL/6-Tg(APOA1)1Rub/J (The Jackson Laboratory). Control mice were C57BL/6, which express murine apoA-I. 3 d after injections and every other day thereafter blood samples (20 μl) were taken from the animals via tail bleeds for evaluation of secreted human proteins.

### Example 3

### Purification of Lipoproteins

HDL was purified from the plasma of two mice by adjusting the density to 1.25 g/ml with KBr and centrifuged for 16 h at 49,000 rpm at 10° C. in NVTi65 rotor. The lipoprotein fractions were collected, concentrated, and fractionated on a Superdex 200 HR 10/30 column (GE Healthcare) (Lugli, et al., “Characterization of Primate Trypanosome Lytic Factors,” Mol. Biochem. Parasitol. 138:9-20 (2004), which is hereby incorporated by reference in its entirety).

### Example 4

### Electrophoresis and Immunoblotting

Plasma samples or lipoprotein fractions were separated on 7.5% Tris-glycine PAGE Gold precast gels (Cambrex), transferred onto PDVF membranes (GE Healthcare), and probed with the following: polyclonal rabbit anti-human haptoglobin (1:20,000; Sigma-Aldrich); mouse monoclonal anti-Hpr (1:5,000); mouse monoclonal anti-apoL-I (1:10,000), polyclonal rabbit anti-human apoL-I (1:10,000; Proteintech Group, Inc.), polyclonal goat anti-human apoL-I (N20, 1:100; Santa Cruz Biotechnology), polyclonal sheep anti-human apoA-I (1:10,000; AbD Serotec), and polyclonal rabbit anti mouse apoA-I (1:10,000: Abcam). Secondary antibodies conjugated to horseradish peroxidase used were as follows: anti-rabbit IgG (1:10,000), anti-mouse IgG (1:50,000), anti-goat IgG (1:10,000; all Promega), and anti-sheep IgG (1:10,000; Roche).

### Example 5

### Trypanosomes

The following trypanosomes were used: serum-sensitive T. b. brucei ILTat1.25 and T. b. brucei Lister 427-derived cell line, the serum-resistant strain T. b. brucei 427-derived cell line expressing the serum resistance-associated (SRA) gene (Wang, et al., “Structural Features Affecting Variant Surface Glycoprotein Expression in Trypanosoma brucei,” Mol. Biochem. Parasitol. 128:135-145 (2003), which is hereby incorporated by reference in its entirety), serum-sensitive T. evansi (Antat 3), T. b. rhodesiense KETRI 243, and T. congolense (STIB68 Q).

### Example 6

### In Vivo and in Vitro Experiments

For survival experiments mice were infected i.p. with 1.78×10⁶ or 5,000 trypanosomes on day 3 after high-volume injection of the human genes. For plasma transfer experiments, on day 3 after transfection with human transgenes, mice were killed. Aliquots of 300 μl of plasma were injected i.v. into naive mice that were infected with T. b. brucei (0.5-2.5×10⁸/ml). Parasitemia was followed, and mice that reached parasitemia of 1×10⁹/ml were killed and their time of death was recorded.

Trypanosomes (T. b. brucei ILTat 1.25; 10⁶) or T. b. brucei-SRA 427 were incubated for 150 min at 37° C. in DME, 0.2% BSA with aliquots of sized fractionated lipoproteins. Where indicated, parasites were incubated with NH₄Cl (10 mM). Parasite lysis was determined using a calcein-AM fluorescence-based assay (Tomlinson, et al., “High-density-lipoprotein-independent Killing of Trypanosoma brucei brucei by Human Serum,” Mol. Biochem. Parasitol. 70:131-138 (1995), which is hereby incorporated by reference in its entirety).

### Example 7

### Creation of TLF Mice Using Hydrodynamic-Based Gene Delivery

To understand the role of Hpr, apoL-I, and Tr-apoL-I and to explore reconstitution of TLF activity in vivo, a physiologically relevant system that allows transgenic expression of individual or multiple TLF components in a mouse was created. Using a hydrodynamic gene-based delivery approach (Kobayashi, et al., “Hydrodynamics-based Procedure Involves Transient Hyperpermeability in the Hepatic Cellular Membrane: Implication of a Nonspecific Process in Efficient Intracellular Gene Delivery,” J. Gene Med. 6:584-592 (2004), which is hereby incorporated by reference in its entirety), Tg mice that express native Hpr, apoL-I or Tr-apoL-I predominantly from the liver were generated (Song, et al., “Hydrodynamics-based Transfection: Simple and Efficient Method for Introducing and Expressing Transgenes in Animals by Intravenous Injection of DNA,” Methods Enzymol. 346:92-105 (2002), which is hereby incorporated by reference in its entirety), which is the normal site where TLF components are synthesized and assembled. Expression plasmids that encode Hpr and/or apoL-I or Tr-apo L-I are expressed and proteins are secreted into the circulation within 24 h.

Human TLF has several well-characterized properties that define it as a specific subset of HDL. The following criteria were used as a benchmark for comparing the activity of reconstituted TLFs from Tg mice: a) co-immunoprecipitation experiments have revealed that Hpr and apoL-I are coexpressed in the same particle despite constituting only 1% of HDL molecules, suggesting that their distribution in HDL particles is nonrandom (Shiflett, et al., “Human High Density Lipoproteins are Platforms for the Assembly of Multi-component Innate Immune Complexes,” J. Biol. Chem. 280:32578-32585 (2005) and Widener, et al., “Hemoglobin is a Co-factor of Human Trypanosome Lytic Factor,” PLoS Pathog. 3:e129 (2007), which are hereby incorporated by reference in their entirety); b) Trypanolytic activity of human plasma or purified TLF against T. b. brucei can be demonstrated in an in vitro killing assay during which the trypanosomes swell and burst. Alternatively, human plasma can be i.v. injected into a mouse to provide protection against T. b. brucei infection (Raper, et al., “Trypanosome Lytic Factors: Novel Mediators of Innate Immunity,” Curr. Opin. Microbiol. 4:402-408 (2001), which is hereby incorporated by reference in its entirety); c) Trypanosomes that express SRA either naturally (T. b. rhodesiense) or artificially (T. brucei-SRA) are resistant to trypanolysis by TLF in vitro (Vanhamme, et al., “Apolipoprotein L-I is the Trypanosome Lytic Factor of Human Serum,” Nature 422:83-87 (2003), which is hereby incorporated by reference in its entirety) or in vivo after transfer of human plasma or purified TLF i.v. to mice (Raper, et al., “Trypanosome Lytic Factors: Novel Mediators of Innate Immunity,” Curr. Opin. Microbiol. 4:402-408 (2001), which is hereby incorporated by reference in its entirety); d) Trypanolytic activity in vitro can be blocked by addition of weak bases to prevent acidification of internal vesicles, which is required to activate TLF after uptake and internalization of the lipoprotein particle by the parasite (Hager, et al., “Endocytosis of a Cytotoxic Human High Density Lipoprotein Results in Disruption of Acidic Intracellular Vesicles and Subsequent Killing of African Trypanosomes,” J. Cell Biol. 126:155-167 (1994), which is hereby incorporated by reference in its entirety).

### Example 8

### Apolipoprotein L-I Protects Mice from Infection with T. B. Brucei, T. Evansi, and T. Congolense

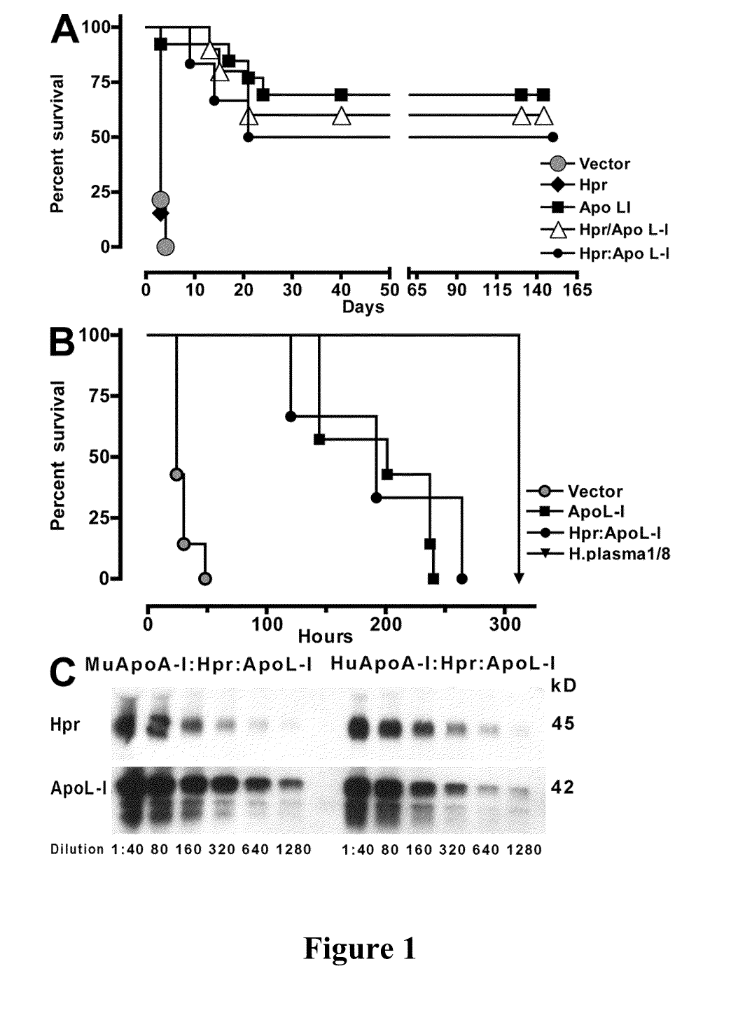

Tg mice expressing genes that encode for Hpr and apoL-I individually reveal that only mice expressing the apoL-I transgene (Tg-apoL-I) are protected from challenge with T. b. brucei. In contrast, mice that express the Hpr transgene (Tg-Hpr) are not protected and succumb to infection with the same kinetics as mice given the empty vector (FIG. 1A). It was then evaluated whether the expression of Hpr and apoL-I together in vivo would give additional protection beyond that seen with apoL-I. There is no increase in protection when the mice express both Hpr and apoL-I either from individual plasmids (Tg-Hpr/apoL-I) or coexpress them from a single plasmid (Tg-Hpr:apoL-I), which allows protein synthesis in the same transfected cell. These results suggest that in vivo apoL-I is the main lytic component of TLF.

Tg-apoL-I mice mimic humans in their resistance and sensitivity to infection with a variety of trypanosome species (Table 1).

TABLE 1
Transgenic Mice that Express the ApoL-I Gene Ameliorate Infection
with T. brucei, T. evansi, and T. congolense.

| | | Number mice alive/ | | |
|---|---|---|---|---|
| Transgenic | Trypanosome species | number mice infected | | |
| DNA | (1.78 × 10⁶ injected i.p.) | Day 3 | Day 20 | Day 50 |
| ApoL-I | T. brucei | | 5/5 | 3/5^{b} |
| Vector | | 0/5^{a} | | |
| ApoL-I | T. brucei SRA | 0/5^{a} | | |
| Vector | | 0/5^{a} | | |
| ApoL-I | T. evansi | | 5/5 | 4/5^{c} |
| Vector | | | 5/5 | 1/5^{d} |
| ApoL-I | T. congolense | | 5/5 | 3/5^{b} |
| Vector | | | 2/5 | 2/5^{d} |

Mice expressing the gene for apoL-I were infected with 1.78 × 10 T. b. brucei (Lister 427-derived line), T. b. brucei-SRA (Lister 427-derived line expressing the SRA gene), T. evansi (AnTat 3), or T. congolense (STIB 68Q). n = 5 per group.
^{a}Parasitemia ≧1 × 10⁹ before mice were killed.
^{b}No parasites detectable
^{c}Died of unknown causes, did not have detectable parasites.
^{d}Parasitemia = 1 × 10⁶/ml

Nonprimate mammals do not have the genes that encode for Hpr and apoL-I, and, therefore, they do not produce TLF. They are thus susceptible to a broader range of trypanosome species than humans, such as T. congolense, T. evansi, and T. b. brucei, which limits their survival in Central Africa. T. congolense and T. evansi are both trypanosomes whose course of infection is mild and prolonged because of relapsing but ultimately lethal parasitemias, whereas the T. b. brucei used in these experiments has a short course of infection that is rapidly lethal. It is shown here that Tg-apoL-I mice can ameliorate infections with all these trypanosome species and all Tg-apoL-I survivors are parasite-free 20 d after infection (Table 1). As expected, Tg-apoL-I mice infected with T. brucei-SRA were unable to control the parasitemia, and all mice succumbed to infection within 3 days with kinetics identical to mice that received the empty vector (Table 1).

Transgene expression decreases with time in the Tg mice; blood levels of apoL-I begin to decrease around day 14 and Hpr levels begin to decrease around day 7, but low levels of protein can still be detected by day 45. Consequently, sometimes it is observed that a recrudescence of parasites occurs at 20-25 d after infection, which can develop into a highly lethal parasitemia (10⁹/ml). If there is no detectable parasitemia by day 25 in the Tg mice, they will remain parasite-free indefinitely. These parasites that recrudesce are not resistant to TLF, and, therefore, it can be concluded that some parasites are in tissue spaces to which TLF has no access, or that the TLF titer is inadequate to kill all of the trypanosomes.

Although Tg-apoL-I mice exhibit many of the properties of human TLF, experiments in which killing activity was assessed by plasma transfer to naive mice indicate that expression of apoL-I gives lower trypanolytic activity than that observed for human plasma. Transfer of 300 μl of Tg-apoL-I plasma to naive mice already infected with T. b. brucei delayed the time to peak parasitemia (10⁹/ml) with a median survival of 201 h (FIG. 1B). Despite the variability in the plasma transfer assay, it is clear that plasma from both Tg-apoL-I (median survival 201 h) and Tg-Hpr:apoL-I (median survival 192 h) confer equal protection, which indicates that coexpression of Hpr in the context of murine HDL does not increase lytic activity.

Transfer of 300 μl human plasma completely cleared the trypanosome infection and protected the mice indefinitely. To gauge the relative amount of lytic units within the plasma from different Tg mice, human plasma was serially diluted until survival time was ˜300 h (1:8, 37 μl; FIG. 1B). In this plasma transfer assay, it was found that Tg mouse plasma has ˜10% the lytic capacity of normal human plasma. In contrast to human plasma, Tg-apoL-I plasma or Tg-Hpr:apoL-I plasma showed no trypanolytic activity in vitro within the time frame of the standard assay, which suggests that the Tg plasma is missing some additional human component that augments or stabilizes activity in vitro or in vivo.

### Example 9

### Human apoA-I and Haptoglobin-Related Protein Increase the Specific Activity of HDL Synthesized by Tg Mice

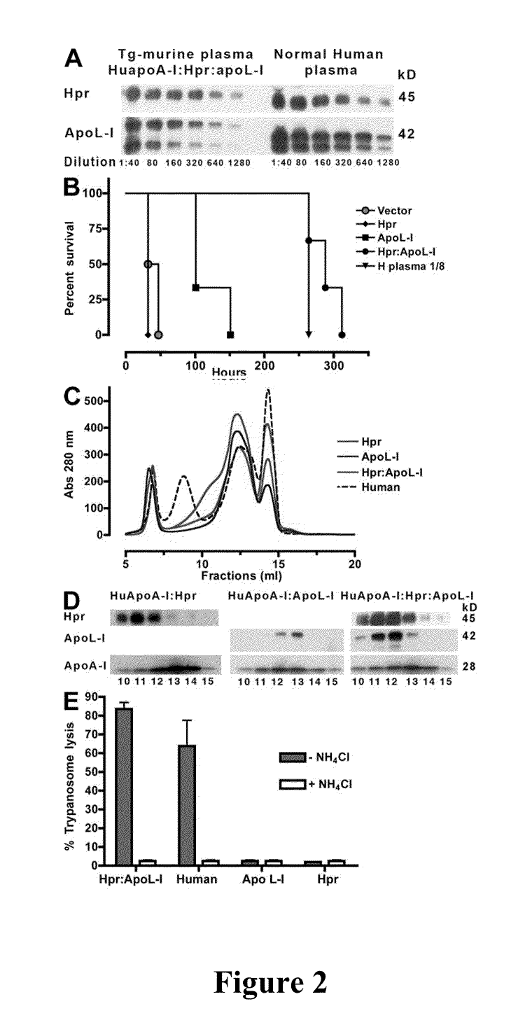

The ability of human apoA-I to augment or stabilize the Tg-TLF activity in vitro or in vivo was evaluated. In contrast to mice, humans have three major HDL subfractions designated HDL1˜11.4 nm, HDL2˜10.2 nm, and HDL3˜8.7 nm. Mice containing a stably integrated human apoA-I transgene (HuapoA-I) express ˜2 mg/ml human apoA-I and 0.1 mg/ml murine apoA-I and exhibit a humanlike HDL profile of HDL2 and HDL3, indicating that the amino acid sequence of apoA-I is a determinant of the HDL distribution (Rubin, et al., “Expression of Human Apolipoprotein A-I in Transgenic Mice Results in Reduced Plasma Levels of Murine Apolipoprotein A-I and the Appearance of Two New High Density Lipoprotein Size Subclasses,” Proc. Nat'l. Acad. Sci. USA. 88:434-438 (1991), which is hereby incorporated by reference in its entirety). Plasma from HuapoA-I mice transfected with Hpr and apoL-I (Hpr:apoL-I) display similar levels of protein either compared with plasma from MuapoA-I mice transfected with Hpr and apoL-I (FIG. 1C) or compared with human plasma (FIG. 2A), corresponding to ˜5-10 μg/ml of apoL-I (Duchateau, et al., “Apolipoprotein L, A New Human High Density Lipoprotein Apolipoprotein Expressed by the Pancreas. Identification, Cloning, Characterization, and Plasma Distribution of Apolipoprotein L,” J. Biol. Chem. 272:25576-25582 (1997), which is hereby incorporated by reference in its entirety) and ˜20-30 μg/ml of Hpr (Muranjan, et al., “Characterization of the Human Serum Trypanosome Toxin, Haptoglobin-related Protein,” J. Biol. Chem. 273:3884-3887 (1998), which is hereby incorporated by reference in its entirety).

In contrast to the murine apoA-I plasma transfer experiments, it was found that expression of apoL-I in the presence of human apoA-I results in plasma that is less lytic than that obtained when both TLF components (Hpr:apoL-I) are expressed. Plasma transfer from HuapoA-I mice transfected with Hpr to naive mice infected with T. b. brucei show no protection by Hpr (median survival 32 h), equivalent to vector alone (median survival 39.5 h; FIG. 2B).

Plasma from HuapoA-I:Hpr:apoL-I transferred to naive mice infected with T. b. brucei has an increase in lytic capacity (median survival time 288 h) compared with that observed with HuapoA-I:apoL-I (median survival 101 h; FIG. 2B). These data reveal that coexpression of Hpr in the context of human apoA-I increases lytic activity, which suggests that human apoA-I may augment or stabilize Tg-TLF (Hpr:apoL-I) activity in vivo. Despite an increase in activity, the triple Tg mouse plasma (HuapoA-I:Hpr:apoL-I) is not as potent as human plasma in transfer experiments. Additional factors present in human plasma, such as TLF2, which is an immune complex of polyclonal IgM and lipid-poor TLF1 that circulates in plasma at ˜10 μg/ml (Raper, et al., “Characterization of a Novel Trypanolytic Factor in Human Serum,” Infect. Immun. 67:1910-1916 (1999), which is hereby incorporated by reference in its entirety), could explain the difference in lytic capacity.

To evaluate the assembly of Hpr and apoL-I into HDLs and the lytic capacity of various Tg mice, all of the different Tg-HDLs containing human apoA-I were purified by density gradient ultracentrifugation followed by size fractionation and, then, each fraction was analyzed by Western blot for localization of Hpr, apoL-I, and apoA-I. Size fractionation of lipoproteins of the HuapoA-I mice show a broad HDL peak (plain lines, #10-14; FIG. 2C), which matches the distribution of human HDL (dashed line). When mice were transfected with a plasmid that encodes for Hpr, it was found that Hpr-HDL is enriched in fractions 11 and 12 (FIG. 2D). When mice were transfected with a plasmid that encodes for apoL-I, it was found that apoL-I-HDL is enriched in fractions 12 and 13 (FIG. 2D). When a single plasmid that encodes both genes under the control of individual promoters to drive expression in the same cell was transfected, it was found that apoL-I has been “redistributed” upon coexpression with Hpr predominantly into fractions 11 and 12 (FIG. 2D). This suggests that when coexpressed, Hpr and apoL-I localize to the same HDL particle. It was observed that a similar redistribution of apoL-I occurs upon coexpression of Hpr in murine apoA-I HDLs.

Purified human TLFs containing either Hpr or apoL-I have low specific activities for in vitro trypanolysis relative to TLFs that contain both Hpr and apoL-I in the same particle (Shiflett, et al., “Human High Density Lipoproteins are Platforms for the Assembly of Multi-component Innate Immune Complexes,” J. Biol. Chem. 280:32578-32585 (2005), which is hereby incorporated by reference in its entirety). Notably, in the experimental system of the present invention, only the purified triple Tg-HDL, which is composed of HuapoA-I:Hpr:apoL-I, showed trypanolytic activity in vitro within the time frame of the standard assay (FIG. 2E). The lytic capacity of the triple Tg-HDL was equivalent to that of human TLF-enriched HDL (both isolated by the same method). In addition, trypanosome lysis by both HDLs was inhibited by incubation of trypanosomes with ammonium chloride (10 mM), a lysosomotropic agent that blocks the acidification of internal organelles (FIG. 2E). This indicates that a functional Tg TLF (HuapoA-I:Hpr:apoL-I) has been created that mimics the properties of human TLF. These data reveal that human apoA-I contributes to an increase in activity of Tg-Hpr:apoL-I HDLs (but not of Tg-apoL-I) that is measurable in plasma transfer experiments in vivo (FIG. 2B), and more so in lytic assays in vitro (FIG. 2E). There was no measurable activity in mouse apoA-I:Hpr:apoL-I HDL in vitro. Therefore, it is concluded that the increase in specific activity observed previously in vitro (Vanhollebeke, et al., “Distinct Roles of Haptoglobin-related Protein and Apolipoprotein L-I in Trypanolysis by Human Serum,” Proc. Nat'l. Acad. Sci. USA. 104:4118-4123 (2007), which is hereby incorporated by reference in its entirety) is caused not only by the presence of just Hpr and apoL-I, but by an interaction between the three human proteins apoA-I, Hpr, and apoL-I (FIGS. 2B and E).

The difference in the specific activity of the Tg-HDLs could arise from changes in the assembly and stability of the HDL complex or changes in the trafficking of Tg-HDLs in the trypanosome. No difference were detected in the assembly of TLFs generated with mouse or human apoA-I based on the following observations. Hpr and apoL-I are exclusively found associated with HDL. Consistently, redistribution, and sometimes an increase, in concentration of apoL-I upon coexpression with Hpr is detected. This suggests that there is an interaction of Hpr with apoL-I. Purification of the Tg-Hpr:apoL-I HDLs by KBr ultracentrifugation and size chromatography gave equivalent yields of Hpr and apoL-I. This suggests that there was no loss of protein components during the purification of TLFs generated with mouse or human apoA-I. Given that no difference in the relative binding to trypanosomes of purified human HDL subclasses containing either Hpr or apoL-I or both Hpr and apoL-I has been reported (Shiflett, et al., “Human High Density Lipoproteins are Platforms for the Assembly of Multi-component Innate Immune Complexes,” J. Biol. Chem. 280:32578-32585 (2005), which is hereby incorporated by reference in its entirety), it is hypothesized that human apoA-I in the presence of Hpr and apoL-I effects either the trafficking of the purified triple Tg HDL or there is resistance to trypanosomal proteases within the lysosome, which is in agreement with others (Shimamura, et al., “The Lysosomal Targeting and Intracellular Metabolism of Trypanosome Lytic Factor by Trypanosoma brucei brucei,” Mol. Biochem. Parasitol. 115:227-237 (2001), which is hereby incorporated by reference in its entirety).

### Example 10

### Apolipoprotein L-I Devoid of the “SRA-Interacting Domain” Does Not Protect Mice from Trypanosome Infection

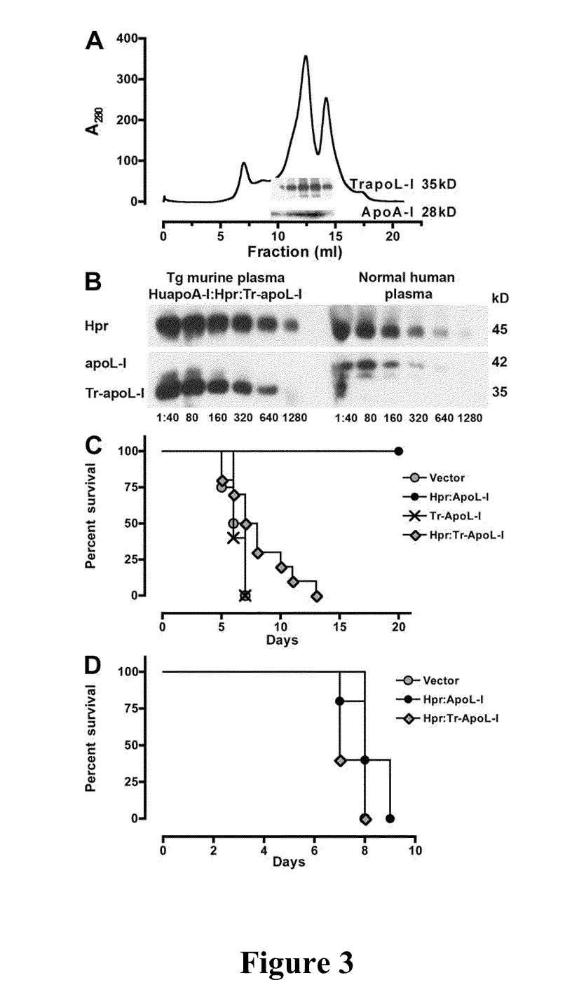

The applicants have established that transient transgenesis provides the first animal model capable of validating reconstituted TLF activity in vivo. The trypanolytic potential of a truncated apoL-I devoid of the SRA-interacting domain (Tr-apoL-I) was next assessed. Full-length apoL-I can kill T. brucei, but not T. brucei that expresses SRA. Tr-apoL-I, which is constructed by deletion of the C-terminal α-helix starting at amino acid 340, has been synthesized in bacteria and cell-free systems and is reported to kill SRA-expressing trypanosomes both in vitro (Vanhamme, et al., “Apolipoprotein L-I is the Trypanosome Lytic Factor of Human Serum,” Nature 422:83-87 (2003), which is hereby incorporated by reference in its entirety) and in vivo when conjugated to nanobodies directed against VSG carbohydrate epitopes and injected into T. brucei-SRA-infected mice (Baral, et al., “Experimental Therapy of African Trypanosomiasis with a Nanobody-conjugated Human Trypanolytic Factor,” Nat. Med. 12:580-584 (2006), which is hereby incorporated by reference in its entirety). Transgenic expression of Tr-apoL-I in livestock could therefore conceivably create animals that are resistant to infection by all species of trypanosomes. Tr-apoL-I-transfected mice revealed robust expression of Tr-apoL-I that readily assembled into HDL particles in a manner similar to full-length apoL-I (FIG. 3A). Surprisingly, the applicants were unable to detect any lytic activity in vivo against SRA-expressing trypanosomes, or even against the susceptible T. b. brucei (Table 2).

TABLE 2
Transgenic Mice that Express the Truncated ApoL-I Devoid
of the SRA-interacting Domain do not Protect from Infection
with T. b. brucei or T. b. brucei-SRA

| | | Number mice alive/ | |
|---|---|---|---|
| Transgenic | Trypanosome species | number mice infected | |
| DNA | (1.78 × 10⁶ injected i.p.) | Day 3 | Day 50 |
| ApoL -I | T. b. brucei | 5/5 | 3/5^{a} |
| Tr-apoL-I | | 0/5^{b} | |
| Vector | | 0/5^{b} | |
| ApoL-I | T. b. brucei SRA | 0/5^{b} | |
| Tr-ApoL-I | | 0/5^{b} | |
| Vector | | 0/5^{b} | |

Tg mice expressing full-length apoL-I or truncated apoL-I infected with 1.78 × 10⁶ T. b. brucei (Lister 427-derived line) or T. b. brucei-SRA (Lister 427-derived line expressing the SRA gene). n = 5 per group.
^{a}No parasites detectable
^{b}Parasitemia ≧1 × 10⁹/ml before mice were killed.

To improve the potential activity of Tr-apoL-I, a double plasmid (Hpr:Tr-apoL-I) was generated and transfected in Tg-HuapoA-I mice, thereby creating HuapoA-I:Hpr:Tr-apoL-I mice. Serial dilution of Tg mice plasma revealed robust expression of both proteins (FIG. 3B), which could be co-immunoprecipitated, indicating their localization in the same HDL. However, inoculation of mice with a low dose of 5,000 parasites did not reveal any significant trypanolytic activity (P>0.05) of HuapoA-I:Hpr:Tr-apoL-I against T. b. brucei (FIG. 3C) or T. b. brucei-SRA (FIG. 3D).

The discrepancy between active Tr-apoL-I generated in vitro and inactive Tr-apoL-I generated in vivo could be caused by potential differences in conformation, binding, trafficking, and accumulation within trypanosomes. It is unknown how apoL-I folds in solution when synthesized in bacteria in vitro compared with that present in HDL molecules when synthesized in transgenic mice in vivo, but it is likely that the structures are different. Lipid-poor apolipoproteins, depending on the concentration, will form oligomers in physiological media, whereas apolipoproteins bound to lipid-rich HDL particles do not (Davidson, et al., “The Structure of Apolipoprotein A-I in High Density Lipoproteins,” J. Biol. Chem. 282:22249-22253 (2007), which is hereby incorporated by reference in its entirety). The recombinant “lipid-free” Tr-apoL-I-nanobody binds to VSG all over the surface of the parasite, whereas HDL and TLF bind to specific receptors in the flagellar pocket (Drain, et al., “Haptoglobin-related Protein Mediates Trypanosome Lytic Factor Binding to Trypanosomes,” J. Biol. Chem. 276:30254-30260 (2001), Widener, et al., “Hemoglobin is a Co-factor of Human Trypanosome Lytic Factor,” PLoS Pathog. 3:e129 (2007), which are hereby incorporated by reference in their entirety). TLF is trafficked to lysosomes, wherein it is activated (Shiflett, et al., “African Trypanosomes: Intracellular Trafficking of Host Defense Molecules,” J. Eukaryot. Microbiol. 54:18-21 (2007), which is hereby incorporated by reference in its entirety). It is unknown where the “lipid free” Tr-apoL-I nanobody traffics, accumulates, or acts (Engstler et al., “Hydrodynamic Flow-mediated Protein Sorting on the Cell Surface of Trypanosomes,” Cell 131:505-515 (2007), which is hereby incorporated by reference in its entirety).

Despite the lack of activity of Tr-apoL-I, full-length apoL-I bound to HDL and is completely effective in vivo (Table 1 and FIG. 1). Coexpression with human apoA-I and Hpr increases the lytic activity of full-length apoL-I that is measurable in vivo, by plasma transfer (FIG. 2B), and in vitro (FIG. 2E). In contrast, there is no significant activity of Tr-apoL-I bound to HDL, even when coexpressed with human apoA-I and Hpr (FIG. 3C,D). Therefore, the data indicate that the C terminus of apoL-I clearly contributes to lytic activity and is absolutely required when associated with HDL.

A patient from India was diagnosed with T. evansi, which is not normally infective for humans (Joshi, et al., “Human Trypanosomiasis Caused by Trypanosoma evansi in India: The First Case Report,” Am. J. Trop. Med. Hyg. 73:491-495 (2005), which is hereby incorporated by reference in its entirety). The patient had two mutated apoL-I alleles that prevented the production of functional apoL-I protein. It was speculated that the lack of the apoL-I pore-forming domain (stop codon at amino acid 149) or membrane-addressing domain (stop codon at amino acid 268) were key to the patient's susceptibility to T. evansi (Vanhollebeke, et al., “Human Trypanosoma evansi Infection Linked to a Lack of Apolipoprotein L-I,” N. Engl. J. Med. 355:2752-2756 (2006), which is hereby incorporated by reference in its entirety). In contrast, the Tg-Tr-apoL-I mice data of the present invention show that deletion of the last α-helix at the C terminus (stop codon at 342) is sufficient to eliminate TLF activity in vivo, even though Tr-apoL-I is synthesized and incorporated into HDL and contains both the pore-forming and membrane-addressing domains. Therefore, it is unlikely that a transgene that encodes for this Tr-apoL-I will lead to the production of trypanosome-resistant transgenic animals.

### Example 11

### Discussion for Examples 1-10

Overall, the in vivo data show that apoL-I is necessary and sufficient to kill trypanosomes. Conversely, Tr-apoL-I, which was predicted to be lytic for T. b. brucei-SRA, and, therefore, T. b. rhodesiense, is unable to kill any trypanosomes in vivo or in vitro, thereby underscoring the importance of the C-terminal α-helix of apoL-I in the context of HDL. Hpr expressed in vivo does not cause the lysis of African trypanosomes. Although Tg-Hpr mice have been previously described, no lytic activity was detected in purified HDL in vitro (Hatada, et al., “No Trypanosome Lytic Activity in the Sera of Mice Producing Human Haptoglobin-related Protein,” Mol. Biochem. Parasitol. 119:291-294 (2002), which is hereby incorporated by reference in its entirety), which is in agreement with the data. Applicants have found that the addition of Hpr does not enhance the in vivo protection directly within the Tg mice, over and above apoL-I. Despite the observation that Hpr redistributes apoL-I into higher molecular mass HDLs, Hpr in combination with apoL-I is not sufficient to generate TLF with lytic activity that is measurable in vitro within the time frame of the assay. It is the combination of human apoA-I, Hpr, and apoL-I that recreates a fully functional “human TLF” in vitro, with equivalent lytic capacity to purified human TLF. This data leads to the conclusion that all three human proteins are beneficial in effecting maximal killing and emphasize the importance of Transgenic mice to validate hypotheses generated through in vitro experimentation.

### Example 12

### Experimental Methods for Examples 13-24

Baboon TLF was purified from plasma (Southwest National Primate Research Center) by density gradient centrifugation (Lugli et al., “Characterization of Primate Trypanosome Lytic Factors,” Mol Biochem Parasitol 138:9-20(2004), which is hereby incorporated by reference in its entirety), a two-step gel filtration procedure and affinity chromatography using sepharose-NHS coupled polyclonal anti-Haptoglobin IgG which cross reacts with baboon Hpr. Fractions were tested at each stage for TLF activity using a standard trypanosome lysis assay (Tomlinson, S. et al., “High-density-lipoprotein-independent Killing of Trypanosoma brucei by Human Serum,” Mol Biochem Parasitol 70, 131-8 (1995), which is hereby incorporated by reference in its entirety).

TLF samples separated by 4-20% SDS PAGE were silver stained and proteins were analysed by Q-TOF tandem mass spectrometry (Protein Analysis facility, NYULMC, US and FingerPrints Protein Analysis Facility, University of Dundee, UK). Proteins were identified by database matching (MASCOT) or in the case of baboon apoL-I, by de novo sequence assignment.

Baboon apoL-I peptide sequences were used to clone the apoL-I gene from total baboon liver RNA using 5′ RACE and RT-PCR. A baboon Hpr gene fragment was used to clone the full Hpr gene by 5′ RACE from liver RNA. Baboon apoL-I and Hpr genes were inserted into pRG977 plasmid (either alone or in combination) for expression in mice by hydrodynamic gene delivery (HGD) (Molina-Portela et al., “Distinct Roles of Apolipoprotein Components Within the Trypanosome Lytic Factor Complex Revealed in a Novel Transgenic Mouse Model,” J Exp Med 205:1721-8 (2008); Kobayashi et al., “Hydrodynamics-based Procedure Involves Transient Hyperpermeability in the Hepatic Cellular Membrane: Implication of a Nonspecific Process in Efficient Intracellular Gene Delivery,” J Gene Med 6:584-92 (2004), which are hereby incorporated by reference in their entirety). On day 3 post injection of plasmid, the presence of baboon proteins in blood was verified by western blot and mice were infected with 5000 trypanosomes. The following trypanosomes were used: serum-sensitive T. b. brucei Lister 427-derived cell line, the human serum-resistant strain T. b. brucei 427-derived cell line expressing the serum resistance-associated (SRA) gene (427-SRA) and T. congolense (STIB68 Q). The time of death was recorded (T. b. brucei) or the blood parasitaemia was counted in a haemocytometer (T. congolense). In addition, serum was prepared from day 3 HGD mice and assayed directly for trypanosome lytic activity or plasma HDLs were prepared by density gradient centrifugation and gel filtration and assayed for trypanosome lytic activity.

### Example 13

### Preparation and Analysis of Baboon TLF

Total baboon lipoproteins (˜100 mg of protein) were fractionated on a 100 ml superose-6 gel filtration column. Trypanolytic fractions were identified as previously described (Lugli et al., “Characterization of Primate Trypanosome Lytic Factors,” Mol Biochem Parasitol 138:9-20(2004), which is hereby incorporated by reference in its entirety), pooled, and concentrated. HDL was further separated from serum proteins using a high-resolution Superdex-200 gel filtration column. To obtain highly purified TLF, lytic fractions were applied to a 1 ml polyclonal anti-human haptoglobin IgG (Sigma) coupled NHS sepharose column (GE Lifesciences). The bound fraction, containing TLF was eluted using 0.1 M TEA, pH 11.5 and neutralized with 1 M Tris pH 5.0. Specific activity was assessed at each stage using the standard trypanosome lysis assay (Raper et al., “Characterization of a Novel Trypanosome Lytic Factor from Human Serum,” Infect Immun 67:1910-6 (1999), which is hereby incorporated by reference in its entirety).

### Example 14

### Cloning of the Baboon apoL-I and Baboon Hpr Genes

Baboon liver biopsies were obtained from the Southwest National Primate Research Center, San Antonio Tex. Total RNA was extracted from a baboon (0.5 g) liver using Trizol reagent (Invitrogen), according the manufacturer's instructions. 5′ RACE was performed using the 5′/3′ RACE kit 2^{nd} generation (Roche) using 1 μg total RNA. Primer sequence designs were based on the human apoL-I gene sequence, with alterations consistent with the baboon apoL-I peptide sequences obtained from mass spectrometry:

| SP1 (cDNA synthesis primer): | |
|---|---|
| CTCCAGCTCCTGAGCCAC | (SEQ ID NO: 18) |
| SP2 (gene specific reverse PCR primer): | |
| GCTGAGACTGGCTCAGTGAC. | (SEQ ID NO: 19) |

The 5′ apoL-I cDNA was cloned and the sequence used to query the NCBI nucleotide database. The top hits were a Maccaca mulata BAC clone (GenBank Accession No: gb|AC200580) followed by the human apoL-I gene. The macaque sequence was aligned with the human apoL-I gene to reveal a near identical sequence in the 3′ UTR, which was used to design a reverse primer for RT PCR of the entire baboon apoL-I coding sequence:

Forward:
(SEQ ID NO: 20)
CTCGAGGCCACCATGGAGGGAGCTGCTTTGCTGAGAC.
[Unique Xho-I site (italic) and kozak sequence
(bold) were added].
Reverse:
(SEQ ID NO: 21)
GGTGGTTGCCCTGCCCTGTGG

A baboon Hpr gene fragment was cloned previously by RT-PCR. The remaining 5′ end was obtained by 5′ RACE using the following specific internal primer:

| | | |
|---|---|---|
| Reverse: | GCATAATCCTTTGAGGGTAGGCAG | (SEQ ID NO: 22) |

### Example 15

### Expression of Baboon apoL-I and Baboon Hpr Genes in Mice Using Hydrodynamic Gene Delivery (HGD)

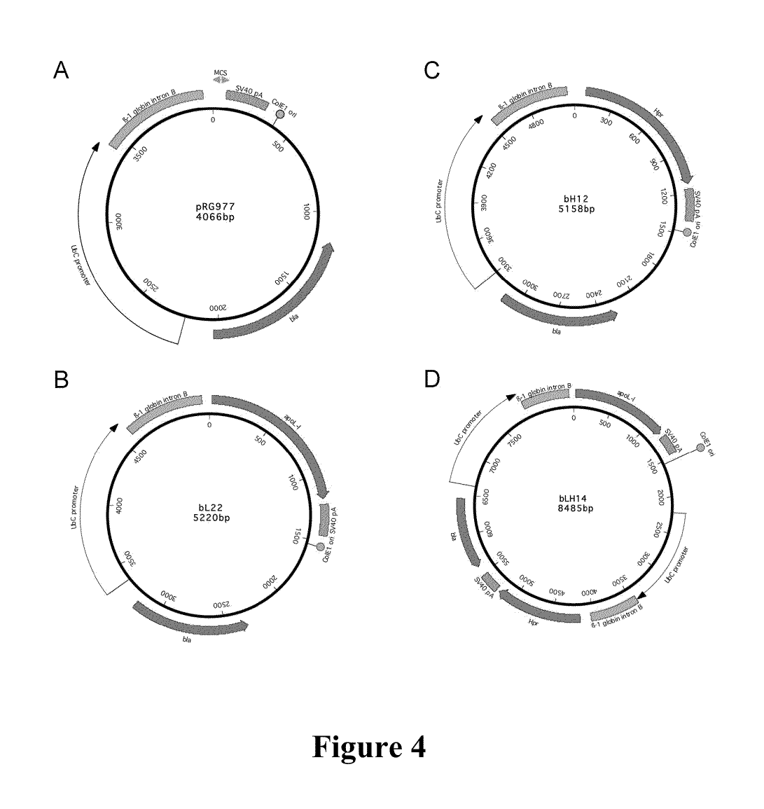

Baboon apoL-I, Hpr or both baboon apoL-I and Hpr coding sequences were inserted into a mammalian expression vector (pRG977) as shown in FIG. 4. All constructs contain a β-lactamase gene and an origin of replication to allow for propagation in bacteria. Each apoL-I or Hpr coding sequence is ligated into a multiple cloning site, positioned between a β-globin intron (5′ of coding sequence) and an SV40 poly(A) signal sequence (3′ of coding sequence) in order to increase the stability and cytoplasmic translocation of the encoded mRNA transcripts in mammalian cells (FIG. 4). To allow the independent expression of baboon apoL-I and baboon Hpr genes in the same mouse cell, a dual expression construct was made in which each gene has a separate ubiquitin promoter, β-globin intron and SV40 poly(A) signal sequence (FIG. 4). Mice were injected i.v. with 50 μg or 100 μg of plasmid for single and dual expression constructs respectively. Plasmids were administered in 2.0-2.8 ml. of saline, which allows for secretion of human apoL-I and Hpr from mouse hepatocytes into blood within 24 hours as described (Molina-Portela et al., “Distinct Roles of Apolipoprotein Components within the Trypanosome Lytic Factor Complex Revealed in a Novel Transgenic Mouse Model,” J Exp Med 205, 1721-8 (2008); Kobayashi et al., “Hydrodynamics-based Procedure Involves Transient Hyperpermeability in the Hepatic Cellular Membrane: Implication of a Nonspecific Process in Efficient Intracellular Gene Delivery,” J Gene Med 6:584-92 (2004), which are hereby incorporated by reference in their entirety). apoL-I gene chimeras were produced using overlap extension PCR, exactly as described previously (Heckman et al., “Gene Splicing and Mutagenesis by PCR-driven Overlap Extension,” Nat Protoc 2: 924-932 (2007), which is hereby incorporated by reference in its entirety). C57BL/6-Tg(APOAI)1Rub/J, congenic C57BL/6 and Swiss Webster mice were used for all in vivo experiments.

### Example 16

### Antibodies and Western Blotting

HGD mouse plasma was obtained on day 3 post injection of plasmid and subjected to Western blot analysis (Molina-Portela et al., “Distinct Roles of Apolipoprotein Components within the Trypanosome Lytic Factor Complex Revealed in a Novel Transgenic Mouse Model,” J Exp Med 205: 1721-8 (2008), which is hereby incorporated by reference in its entirety). The antibodies used were as follows: polyclonal rabbit anti-haptoglobin (Sigma, 1:20,000), polyclonal sheep anti-human apoA-I (1:10,000; AbD Serotec), and polyclonal anti-human apo L-I (1:10,000; ProteinTech). The anti-baboon apoL-I polyclonal (1:500) was obtained from mice immunized intradermally with the baboon apoL-I expression construct used in HGD experiments (Regeneron, Tarrytown, N.Y., USA). Secondary antibodies were horse radish peroxidase conjugated and used as follows: anti-Rabbit IgG (1:20,000; Promega); anti-sheep IgG (1,10000; Roche) and anti-mouse IgG Fcγ (1:5000; Jackson Immuno Research Labs).

### Example 17

### Trypanosomal Strains and Killing Assays

On day 3 post injection of plasmid, the mice were infected with 5000 trypanosomes. The following trypanosomes were used: human serum-sensitive T. b. brucei Lister 427-derived cell line, the human serum resistant strain T. b. brucei 427-derived cell line expressing the serum resistance-associated (SRA) gene (427-SRA), T. congolense (STIB68 Q) and T. b. rhodesiense KETRI 243, isolated from a human patient in Busoga, Uganda. To ensure retention of human serum resistance, KETRI 243 was grown in mice in the presence of 0.3 ml normal human serum injected i.v. The time of death was recorded (T. b. brucei and T. b. rhodesiense) or the blood parasitemia was counted in a hemocytometer (T. congolense).

Serum was prepared from day 3 HGD mice and assayed directly for trypanosome lytic activity or plasma HDLs were prepared by density gradient centrifugation and gel filtration and assayed for trypanosome lytic activity. For serum killing assays trypanosomes (T. b. brucei, strain 427-SRA) were obtained directly from the buffy coat of heavily infected heparinized mouse blood. The trypanosomes were diluted into prewarmed DMEM containing 10% FBS. In 96-well plates, transgenic mouse serum was diluted into DMEM+10% FCS and trypanosomes were added to a final volume of 0.2 ml, containing 2.5×10⁵/ml parasites. Killing was allowed to proceed for 17 hours at 37° C. in a CO₂ equilibrated incubator and living trypanosomes were counted using a heamocytometer. HDL killing assays were performed for 150 min at 37° C. in DMEM, 0.2% BSA using aliquots of sized fractionated lipoproteins. Where indicated, parasites were incubated with NH₄Cl (10 mM). Parasite lysis was determined using a calcein-AM fluorescence-based assay (Tomlinson et al., “High-density-lipoprotein-independent Killing of Trypanosoma brucei by Human Serum,” Mol Biochem Parasitol 70: 131-8 (1995), which is hereby incorporated by reference in its entirety).

### Example 18

### Recombinant Serum Resistance Associated Protein and Binding Experiments

His tagged recombinant SRA (residues 24 to 267) was expressed in E. coli from plasmid p3303, a pET15b derivative. The polypeptide was purified using nickel ion affinity chromatography under standard conditions. SRA binding assays were performed, essentially as described previously (Vanhamme et al., “Apolipoprotein L-I is the Trypanosome Lytic Factor of Human Serum,” Nature 422: 83-87 (2003), which is hereby incorporated by reference in its entirety). 25% normal human serum or HGD mouse serum or human HDL or baboon HDL was mixed with 2.5 μg His-tagged SRA in a final volume of 50 μl, with 0.15 M MES pH 5.8, 0.6 M NaCl, 0.35% CHAPS (binding buffer) and protease inhibitors added (Pierce, EDTA free cocktail). For binding experiments involving purified HDL, detergent was omitted, such that the complex would remain intact. After 2 hours at 4° C., 10 μl of Ni-NTA agarose slurry (Qiagen) was added and the suspension was treated according to the manufacturers instructions. Beads were washed in binding buffer and transferred to clean tubes before adding sample buffer. Samples were separated by SDS-PAGE and transferred to PVDF. Western blots were performed with polyclonal anti-human apoL-I (1:10,000; ProteinTech.) and polyclonal rabbit anti-haptoglobin (Sigma, 1:20,000).

### Example 19

### Elucidation of Baboon TLF

The availability of tissues and plasma from primate centers in the US and the availability of partial baboon genome sequence helped in the elucidation of baboon TLF. Highly enriched TLF was obtained from baboon HDLs using affinity chromatography with an anti-human Hp antibody previously used to successfully immunoprecipitate baboon TLF (Table 3).

TABLE 3
Baboon TLF Purification Table.

| | Total | Total | Specific | |
|---|---|---|---|---|
| | Protein | Activity | Activity | Fold |
| Fraction | (mg) | (Units) | (Units/mg) | Purification |
| Plasma | 7500.00 | 10000.00 | 1.33 | — |
| Lipoproteins ^{a} | 135.00 | 14211.00 | 105.27 | 80.00 |
| Superose 6 ^{b} | 56.25 | 12500.00 | 222.22 | 171.00 |
| Superose 200 ^{b} | 8.70 | 3164.00 | 363.68 | 280.00 |
| Anti-Hp affinity ^{c} | 0.04 | 46.70 | 1111.90 | 856.00 |

¹ Unit of activity was defined as the concentration of each fraction required to yield 50% lysis in a standard trypanosome killing assay (Tomlinson et al., “High-density-lipoprotein-independent Killing of Trypanosoma brucei by Human Serum,” Mol Biochem Parasitol 70: 131-8 (1995), which is hereby incorporated by reference in its entirety).
^{a} Lipoproteins were obtained from baboon plasma by density gradient centrifugation resulting in an apparent increase in total activity, due to removal of haptoglobin, an inhibitor of TLF (Raper et al., “Characterization of a Novel Trypanosome Lytic Factor from Human Serum,” Infect Immun 67, 1910-6 (1999), which is hereby incorporated by reference in its entirety).
^{b} After sequential gel filtration steps, fractions containing peak trypanolytic activity were pooled. Results shown relate to pooled fractions.
^{c} Eluate obtained from anti-Hp polyclonal IgG coupled affinity column.

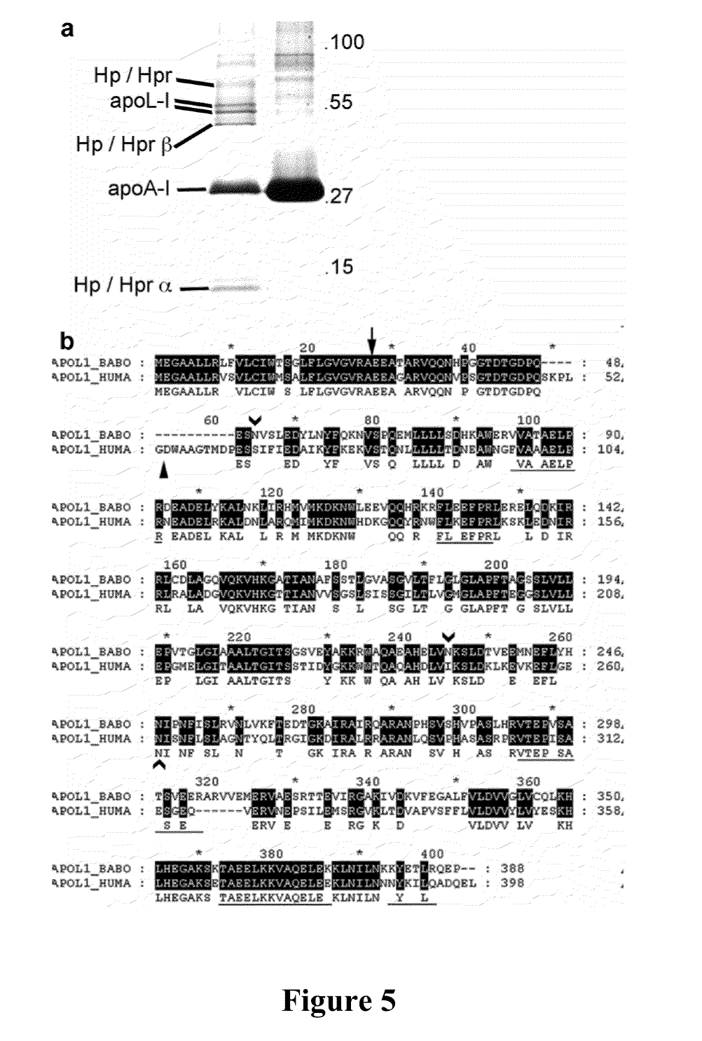

In addition to the canonical HDL apolipoprotein, apoA-I, baboon TLF contains at least 5 unique polypeptides, which were identified by tandem mass spectrometry analysis of tryptic peptides (FIG. 5A and Table 4).

TABLE 4
IQ-TOF Tandem Mass Spectrometry Analysis of Baboon TLF Associated
Tryptic Peptides.

| Protein | Observed | Predicted | | | Amino acid |
|---|---|---|---|---|---|
| ID. | mass | mass | Sequence | SEQ ID NO: | numbers |
| apoA-I | 869.528 | 869.520 | QKVEPLR | 23 | 141-147 |
| | 905.487 | 905.484 | LHELHEK | 24 | 158-164 |
| | 912.471 | 912.453 | AELHEGTR | 25 | 148-155 |
| | 999.553 | 999.547 | LSPLGEEVR | 26 | 165-173 |
| | 1012.584 | 1012.579 | AKPALEDLR | 27 | 231-239 |
| | 1230.716 | 1230.709 | QGLLPVLESFK | 28 | 240-250 |
| | 1249.682 | 1249.704 | DLVTVYVEALK | 29 | 37-47 |
| | 1252.635 | 1252.621 | VQPYLDDFQK | 30 | 121-130 |
| | 1283.570 | 1283.573 | WQEEMELYR | 31 | 132-140 |
| | 1301.649 | 1301.648 | THLAPYSDELR | 32 | 185-195 |
| | 1386.721 | 1386.715 | VSFLSALEEYTK | 33 | 251-262 |
| | 1400.696 | 1400.669 | DYVSQFEGSALGK | 34 | 52-64 |
| | 1427.669 | 1427.662 | KWQEEMELYR | 35 | 131-140 |
| | 1787.852 | 1787.845 | DSGKDYVSQFEGSALGK | 36 | 48-64 |
| | 1932.960 | 1932.934 | EQLGPVTQEFWDNLEK | 37 | 86-101 |
| | 2618.249 | 2618.273 | EQLGPVTQEFWDNLEKETEGLR | 38 | 86-107 |
| Hp(r) | 808.366 | 808.372 | DYAEVGR | 39 | 212-218 |
| | 869.427 | 869.436 | NPADAVQR | 40 | 95-102 |
| | 968.520 | 968.540 | QKVPVNER | 41 | 195-202 |
| | 920.468 | 920.462 | GSFPWQAK | 42 | 112-119 |
| | 923.529 | 923.531 | ILGGHLDAK | 43 | 103-111 |
| | 979.461 | 979.488 | VGYVSGWGR | 44 | 219-227 |
| | 1002.506 | 1002.524 | VMPICLPSK | 45 | 203-211 |
| | 1174.565 | 1174.598 | ATSIQDWVQK | 46 | 333-342 |
| | 1438.607 | 1438.694 | TEGDGVYTLNN EK | 47 | 60-72 |
| | 1708.839 | 1708.850 | LRTEGDGVYTLNN EK | 48 | 58-72 |
| apoL-I | 815.389 | 815.440 | VAQELEK | 49 | 366-372 |
| | 808.401 | 808.440 | KYETLR | 50 | 380-385 |
| | 817.423 | 817.450 | TAEELKK | 51 | 359-365 |
| | 936.431 | 936.470 | FLEEFPR | 52 | 126-132 |
| | 954.503 | 954.550 | VVATAELPR | 53 | 83-91 |
| | 1402.613 | 1402.690 | VTEPVSATSVEER^{a} | 54 | 292-304 |

^{a}Human peptide, VTEPISA (SEQ ID NO: 55) is unique to apoL-I compared to apoL-II-apoL-VI.
Oxidised methionine residue is underlined

Protein bands were identified as Hpr or Hp dimer (60 kDa), Hpr or Hp chains (50 kDa), and Hpr or Hpchains (13 kDa). No peptides were found that distinguished Hpr from Hp, which are ˜96% identical. Two other bands at around 53 kDa did not match any sequences in the database, so peptide sequences were obtained de novo from the mass spectrometry data using the Peaks Online software. Surprisingly, the two bands contained 4 peptide sequences that were homologous to the human apoL gene family; in addition one peptide was unique to apoL-I compared to other apoL paralogues. These data suggest that baboon TLF contains an apoL-I orthologue in addition to Hpr.

To baboon apoL-I and Hpr were further characterized by cloning the full-length genes from baboon liver. The predicted protein product of the baboon apoL-I gene is a 40 kDa mature protein with an additional N-terminal signal peptide that is removed. Baboon apoL-I is 58% identical to the human protein and contains a 14 amino acid deletion and a 6 amino acid insertion (FIG. 5B). There are two potential glycosylation sites in baboon apoL-I compared to a single site in human apoL-I. Baboon Hpr is a predicted 39 kDa mature protein including the N-terminal signal peptide, which remains attached to the secreted protein. It is 94% identical to human Hpr with two extra potential glycosylation sites (Lugli et al., “Characterization of Primate Trypanosome Lytic Factors,” Mol Biochem Parasitol 138:9-20 (2004), which is hereby incorporated by reference in its entirety).

### Example 20

### Role of Baboon apoL-I and Hpr in Killing of Trypanosomes

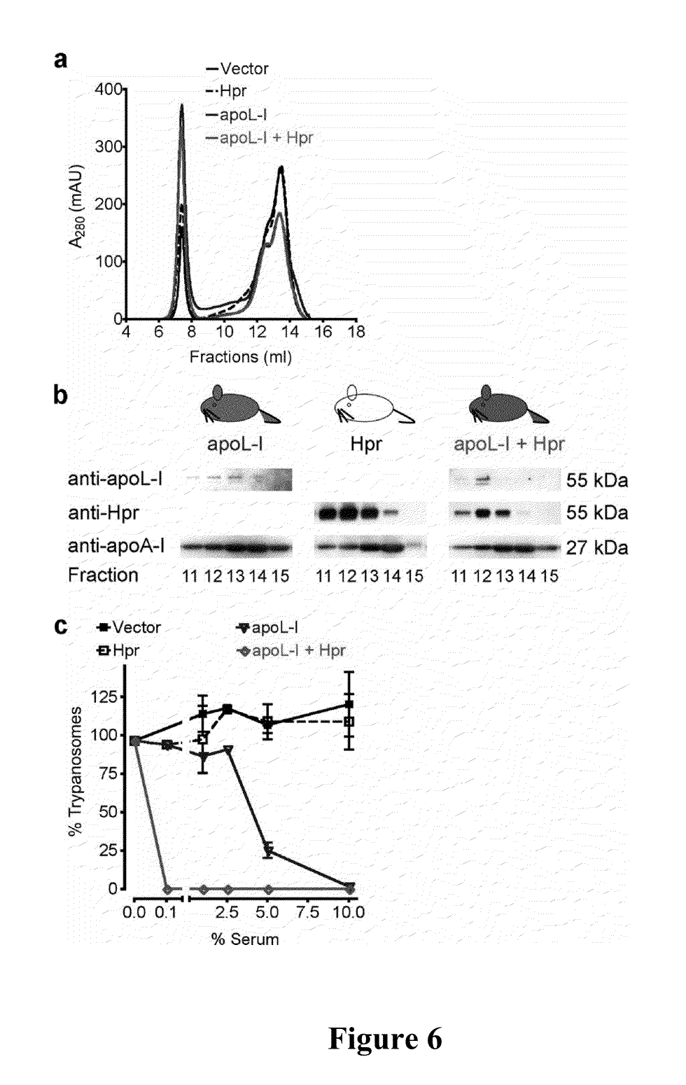

To determine the potential roles of baboon apoL-I and baboon Hpr in the killing of human serum resistant (infective) trypanosomes, hydrodynamic gene delivery (HGD) was used in mice. HGD was recently used to dissect the roles of human TLF components leading to the conclusion that human Hpr, apoL-I and apoA-I are all needed to produce HDLs with comparable lytic activity to human TLF (Molina-Portela et al., “Distinct Roles of Apolipoprotein Components within the Trypanosome Lytic Factor Complex Revealed in a Novel Transgenic Mouse Model,” J Exp Med 205:1721-1728 (2008), which is hereby incorporated by reference in its entirety). Because human apoA-I is 95% identical to baboon apoA-I (compared with ˜65% identity between baboon and murine apoA-I), human apoA-I transgenic mice [Tg(APOA1)] were used. Three days after HGD of baboon Hpr and/or apoL-I, density isolated, size fractionated HDLs from Tg(APOA1) plasma were probed for the presence of baboon TLF proteins by western blotting (FIGS. 6A, B). In the absence of Hpr, baboon apoL-I was broadly distributed throughout the apoA-I containing HDL fractions, whereas in dual transgene expressing mice, baboon apoL-I and Hpr co-segregated into to a relatively high molecular mass HDL sub-fraction. Whether derived from native baboon TLF or from HGD mice, baboon apoL-I migrates on an SDS PAGE gel as a doublet near 55 kDa. This higher than expected apparent molecular mass may arise from glycosylation at one or both of the potential N-linked glycosylation sites in baboon apoL-I.

### Example 21

### Purification, Identification, Cloning and Expression of Baboon TLF Components

In order to understand the mechanism of killing of T. b. rhodesiense by non-human primates baboon TLF was used. It was tested due to the availability of tissues and plasma from primate centres in the US and the availability of a partial baboon genome sequence. Highly enriched TLF was obtained from baboon HDLs using affinity chromatography with an anti-human Hp, previously used to immunoprecipitate baboon TLF (Lugli et al., Characterization of Primate Trypanosome Lytic Factors,” Mol Biochem Parasitol 138: 9-20 (2004), which is hereby incorporated by reference in its entirety) (Table 3). The components of baboon TLF included the canonical HDL apolipoprotein, apoA-I and at least other 5 polypeptides (FIG. 5A), which were identified by tandem mass spectrometry analysis of tryptic peptides (Table 4). The identified polypeptides were: (i) Hpr or Hp dimer (60 kDa), Hpr or Hp β chains (50 kDa), and Hpr or Hp α chains (13 kDa); no peptides were identified that distinguished Hpr from Hp, which are ˜96% identical (Lugli et al., Characterization of Primate Trypanosome Lytic Factors,” Mol Biochem Parasitol 138: 9-20 (2004), which is hereby incorporated by reference in its entirety). (ii) Two other polypeptides of ˜53 kDa did not match any sequences in the database, so peptide sequences were obtained de novo from the mass spectrometry data using Peaks Online. The two polypeptides contained 4 peptide sequences that were homologous to the human apoL gene family; in addition, one peptide was unique to apoL-I compared to other apoL paralogs. These data indicated that baboon TLF contains an apoL-I ortholog in addition to Hpr.

To further characterize baboon apoL-I and Hpr, the full-length genes were cloned from baboon liver. The predicted protein product of the baboon apoL-I gene is a 40 kDa mature protein with an additional N-terminal signal peptide that is removed. Baboon apoL-I is 58% identical to the human protein and contains a 14 amino acid deletion and a 6 amino acid insertion (FIG. 5B). There are two potential glycosylation sites in baboon apoL-I compared to a single site in human apoL-I. Baboon Hpr is a predicted 39 kDa mature protein including the N-terminal signal peptide, which remains attached to the secreted protein. It is 94% identical to human Hpr with two extra potential glycosylation sites (Lugli et al., Characterization of Primate Trypanosome Lytic Factors,” Mol Biochem Parasitol 138: 9-20 (2004), which is hereby incorporated by reference in its entirety).

To determine the potential roles of baboon apoL-I and baboon Hpr in the killing of human infective trypanosomes, hydrodynamic gene delivery (HGD) of expression constructs was used in mice (FIG. 4). HGD was recently used to dissect the roles of human TLF components leading to the conclusion that human Hpr, apoL-I and apoA-I are required to produce TLF with comparable lytic activity to human TLF (Molina-Portela et al., “Distinct roles of apolipoprotein components within the trypanosome lytic factor complex revealed in a novel transgenic mouse model,” J Exp Med 205: 1721-1728 (2008), which is hereby incorporated by reference in its entirety). Human apoA-I is 95% identical to baboon apoA-I (compared to ˜65% identity between baboon and murine apoA-I) therefore human apoA-I transgenic mice Tg(APOAI) were used as a background strain. Three days after HGD of baboon Hpr and/or apoL-I, HDLs were prepared from plasma and probed for the presence of baboon TLF proteins by western blotting (FIG. 6A, B). In the absence of Hpr, baboon apoL-I was broadly distributed throughout the apoA-I containing HDL fractions (blue mouse), whereas in dual transgene expressing mice, baboon apoL-I and Hpr co-segregated into to a relatively high molecular mass HDL sub-fraction (red mouse). Baboon apoL-I migrates as a doublet near 55 kDa whether expressed in baboons or mice; this higher than expected apparent molecular mass may arise from glycosylation at one or both of the potential N-linked glycosylation sites.

### Example 22

### Evaluation of the Lytic Capability of Baboon TLF Components in Vitro

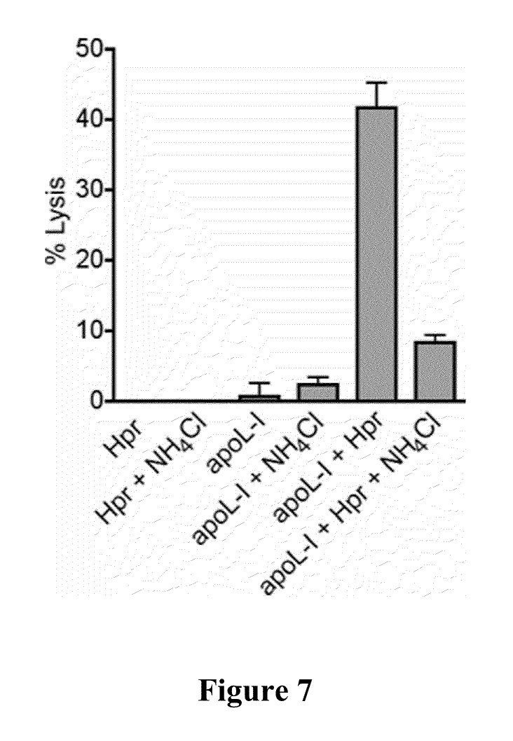

Having confirmed that HGD provided a valid model for synthesizing baboon TLF components, sera and purified HDLs from Tg(APOA1) mice were tested for their capacity to kill human serum resistant trypanosomes (T. brucei 427-SRA) in vitro. Human serum, purified human HDLs or HGD mice expressing human TLF cannot kill human serum resistant (infective) trypanosomes (Molina-Portela et al., “Distinct Roles of Apolipoprotein Components Within the Trypanosome Lytic Factor Complex Revealed in a Novel Transgenic Mouse Model,” J Exp Med 205:1721-8 (2008), which is hereby incorporated by reference in its entirety). Standard 2.5 hr killing assay showed that purified Tg(APOA1) HDL containing baboon apoL-I and Hpr killed human serum resistant (infective) trypanosomes in vitro, whereas there was no measurable activity of purified Tg(APOA1) HDL containing either baboon apoL-I or Hpr. Importantly, the lytic activity of the Tg(APOA1) HDL containing baboon apoL-I and Hpr was inhibited by ammonium chloride, a lysosmotrophic agent that blocks the acidification of internal organelles and thereby blocks TLF activity (FIG. 7). To observe any trace of activity in the sera from the different HGD mice, a long incubation (17 hrs) was performed with human serum resistant (infective) trypanosomes, and a range of sera concentrations (0.1-10%) were tested to discern any differences in specific activity. Tg(APOA1) sera containing baboon apoL-I killed trypanosomes at concentrations of 5% or more, whereas Tg(APOA1) sera from Hpr mice had no activity. The specific lytic activity of Tg(APOA1) sera containing both baboon apoL-I and Hpr was greatly enhanced relative to baboon apoL-I alone, killing at 0.1% (FIG. 6C). These data show that the baboon apoL-I orthologue is responsible for the killing of human serum resistant (infective) trypanosomes and that Hpr enhances this activity.

### Example 23

### Evaluation of the Lytic Capability of Baboon TLF Components in Vivo

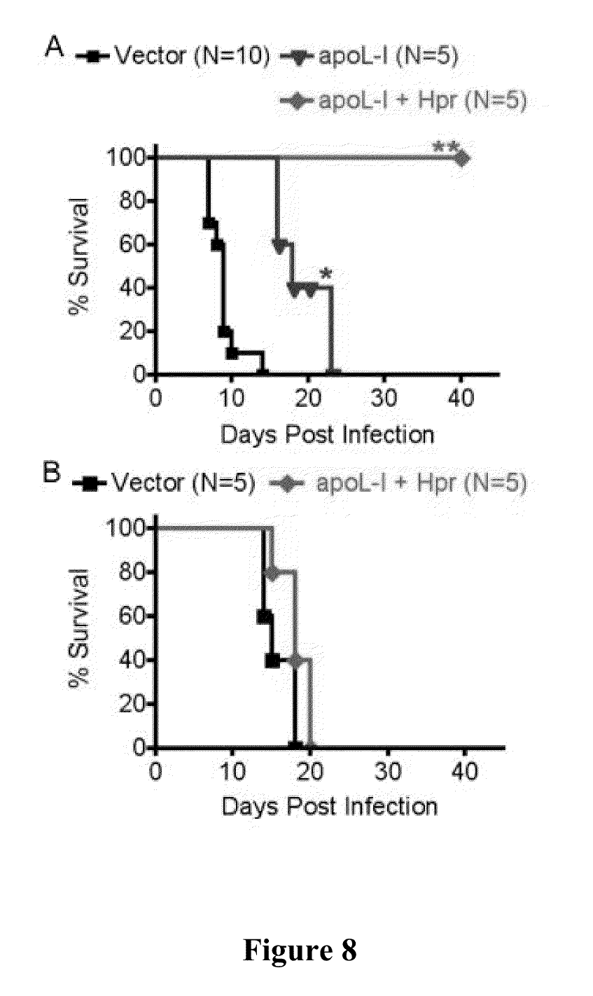

Next, the ability of baboon apoL-I in combination with baboon Hpr to protect against trypanosome infection in vivo was tested. When Tg(APOAI) mice were infected with human serum resistant isolate of T. b. rhodesiense (KETRI 243), originally acquired from a human patient in Busoga, Uganda, the combination of baboon apoL-I and Hpr expression provided complete protection against T. b. rhodesiense infection. Expression of baboon apoL-I alone resulted in an extension of the median survival time by nine days compared to control mice with vector alone (FIG. 8A). This shows that Hpr can increase the lytic capacity of apoL-I. In contrast, the expression of baboon apoL-I and Hpr did not provide protection in the congenic strain of mice (C57BL6), indicating that human apoA-I increased the potency of baboon TLF against T. b. rhodesiense (FIG. 8B).

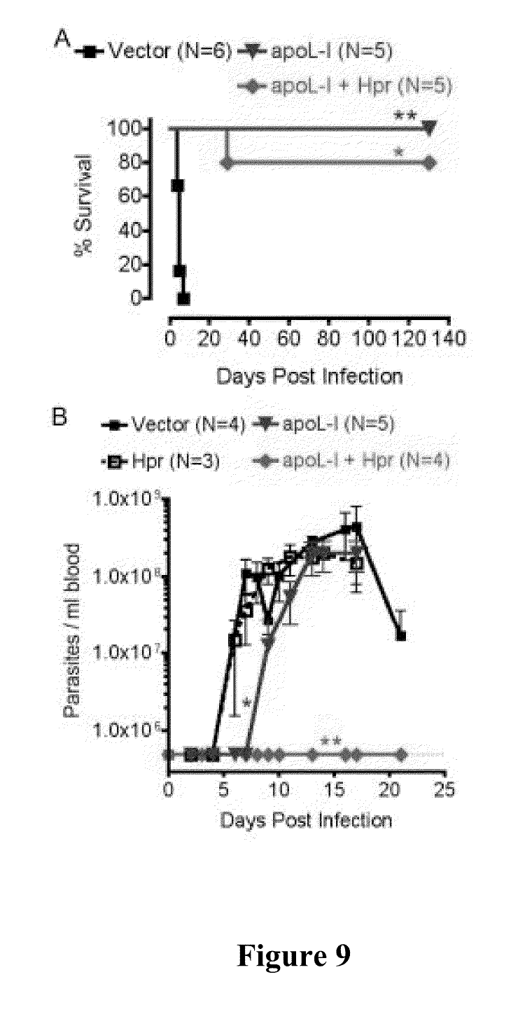

Human serum resistant trypanosomes (T. brucei 427-SRA) were then evaluated, which constitutively express SRA and have a faster doubling time than KETRI 243; control mice died within 4-7 days. Mice expressing apoL-I alone were completely protected whereas, 4 out of 5 mice expressing baboon apoL-I and Hpr were protected (FIG. 9A). The one mouse that developed a parasitemia did so only on day 26 post infection, perhaps due to decreasing levels of apoL-I and Hpr in plasma with time after HGD. It is also possible that the trypanosomes survived in tissue spaces where the HDL (and therefore TLF) concentration is reported to be 25% of that in plasma (Parini, et al., “Lipoprotein Profiles in Plasma and Interstitial Fluid Analyzed with an Automated Gel-filtration System,” Eur J Clin Invest 36:98-104 (2006), which is hereby incorporated by reference in its entirety) and subsequently recrudesced as serum TLF declined. The ability of baboon apoL-I and Hpr to protect against infection with T. congolense was tested, to evaluate the possibility of using baboon TLF to generate transgenic livestock with resistance to other animal infective trypanosomes. T. congolense and T. vivax both produce relapsing and frequently lethal parasitemias in animals. In control mice, infection with T. congolense characteristically resulted in an initial wave of blood parasitemia, which was detectable by day 5 and declined by days 15-20 (FIG. 9B). When baboon apoL-I was expressed in Tg(APOAI) mice there was a delayed parasitemia detectable four days after the control, whereas when both baboon apoL-I and Hpr were expressed in Tg(APOAI) mice there was no detectable parasitemia (FIG. 9B; limit of detection 2×10⁵/ml).

Overall these data suggest that both baboon TLF components may be required to maximally protect transgenic animals from T. congolense and T. b. rhodesiense infection; although sustained production of apoL-I from a stable transgenic may be sufficient. A homologue of the T. brucei Hp/Hpr-Hb receptor, which enhances the uptake of human Hpr-Hb associated TLF complexes has been identified in T. congolense and T. b. rhodesiense (Vahollebeke et al., “A Haptoglobin-hemoglobin Receptor Conveys Innate Immunity to Trypanosoma brucei in Humans,” Science 320:677-681 (2008), which is hereby incorporated by reference in its entirety). This Hp/Hpr-Hb receptor likely increases the uptake and accumulation of baboon TLF in these trypanosomes in vivo.

### Example 24

### Delineation of the SRA Antagonist Domain in Baboon apoL-I

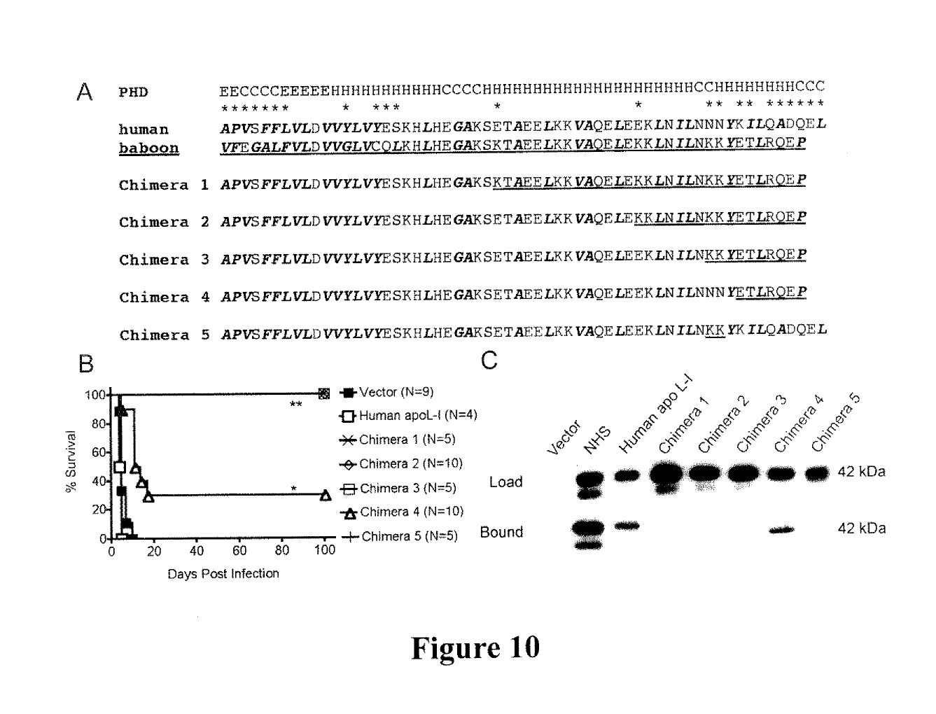

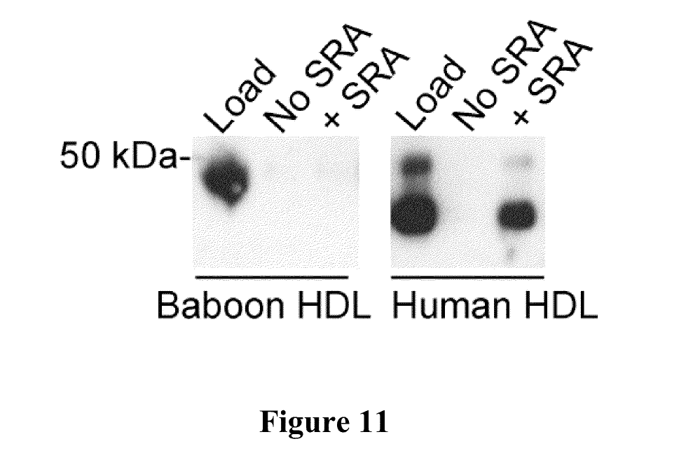

It has been shown that transgenic expression of human apoL-I does not protect against T. b. rhodesiense whereas expression of baboon apoL-I does, and that transgenic protection is lost when the C-terminal domain, which interacts with SRA is deleted (Molina-Portela et al., “Distinct Roles of Apolipoprotein Components within the Trypanosome Lytic Factor Complex Revealed in a Novel Transgenic Mouse Model,” J Exp Med 205: 1721-1728 (2008), which is hereby incorporated by reference in its entirety). Therefore, a series of apoL-I transgenes with human/baboon chimeric C-terminal domains were made (FIG. 10A) and expressed in mice using HGD. Chimeras 1 to 3 protected mice from trypanosomes that expressed SRA whereas chimera 4 did not (FIG. 10B). The difference between chimeras 3 and 4 was just three residues, and within these 3 residues there are two asparagines in human apoL-I versus two lysines in baboon. A chimeric transgene consisting of human apoL-I in which only these two asparagines were substituted with lysines was sufficient to provide complete protection against trypanosomes expressing SRA (FIG. 10A). The ability of the various apoL-I chimeras to kill human serum resistant trypanosomes mirrored the lack of binding to recombinant SRA in vitro (FIG. 10C). Furthermore, human HDL bound SRA whereas baboon HDL did not bind to SRA (FIG. 11).

The present invention discloses a baboon apoL-I ortholog that can transgenically provide protection against animal infective trypanosomes as well as human serum resistant trypanosomes. The ability to kill parasites that express the serum resistance associated protein (SRA), is due to two lysines in the C-terminus of baboon apoL-I, which prevent the binding of apoL-I to SRA.

Lack of binding to SRA may be due to the oligomerization state and/or the charge of the C-terminus of baboon apoL-I. The last 30 amino acids of both human and baboon C-termini form an amphipathic alpha helix, wherein the hydrophobic amino acid face will be buried into the HDL lipid and the hydrophilic face will be exposed. The C-terminus of baboon apoL-I is more basic with a calculated pI of 8.8 and thus will be positively charged and may repel binding to SRA, whereas that of human apoL-I is more acidic with a calculated pI of 4.8, which would be close to neutral in the lysosome. In addition to this charge differential the amphipathic helix contains four heptad repeats ([abcdegf] where a and d are hydrophobic residues) that are predicted to form a coil coiled, which is a superhelical structure stabilized by hydrophobic residues buried in the centre. The baboon C-terminus and the protective chimeras of apoL-I are predicted to form a three stranded coiled coil whereas the human C-terminus is not. It is plausible that trimerization of the C-terminus upon release of the apoL-I from HDL within the lysosome coupled with the surface positive charge prevents the interactions of apoL-I with SRA.

Previous studies revealed that C-terminally truncated human apoL-I, which is unable to bind to SRA, can kill human serum resistant trypanosomes when conjugated to a single domain antibody that binds to a carbohydrate moiety on the surface coat of trypanosomes (Baral et al., “Experimental Therapy of African Trypanosomiasis with a Nanobody-conjugated Human Trypanolytic Factor,” Nat Med 12: 580-584 (2006), which is hereby incorporated by reference in its entirety). Given that the antibody would bind to the entire surface of the trypanosomes and be rapidly and constantly cleared from the surface by hydrodynamic forces and endocytosis (Engstler et al., “Hydrodynamic Flow-mediated Protein Sorting on the Cell Surface of Trypanosomes,” Cell 131: 505-515 (2007), which is hereby incorporated by reference in its entirety), it is surmised that supraphysiological quantities of apoL-I would be delivered to the parasite lysosome by this route. In contrast, mice that express truncated human apoL-I, even when co-expressed with human Hpr and apoA-I, do not have any significant trypanolytic activity; likely due to the delivery of physiological concentrations of apoL-I via Hp-Hb receptor and scavenger receptor mediated endocytosis from the flagellar pocket (Molina-Portela et al., “Distinct roles of apolipoprotein components within the trypanosome lytic factor complex revealed in a novel transgenic mouse model,” J Exp Med 205: 1721-1728 (2008), which is hereby incorporated by reference in its entirety). Thus, truncated human apoL-I can best be described as a hypomorph, it has partial activity under certain conditions but requires the C-terminus for full activity in vivo. In support of this interpretation a recent analysis of the apoL gene family evolution in primates concluded that the C-terminus must be essential otherwise antagonism by T. b. rhodesiense and other pathogens would have resulted in deletion of this domain (Smith et al., “The Apolipoprotein L Family of Programmed Cell Death and Immunity Genes Rapidly Evolved in Primates at Discrete Sites of Host-pathogen Interactions,” Genome Res 19: 850-858 (2009), which is hereby incorporated by reference in its entirety). Instead, parts of the C-terminus have undergone positive selection, in this instance a modification to change the charge, presumably to evade neutralization mechanisms elaborated by pathogens while maintaining the critical function of the C-terminus, which is likely to facilitate proximity to and binding to the target membrane.

Taken together these findings describe the identification of an apoL-I homolog from baboons that can transgenically protect mice from animal infective trypanosomes as well as the human pathogen, T. b. rhodesiense. These findings provide a compelling rationale for creating transgenic livestock expressing baboon apoL-I. The importance of trypanosome resistant cattle is not solely for livestock productivity. In East Africa, Human African Trypanosomiasis is caused by T. b. rhodesiense and is a zoonotic disease with cattle as the major reservoir hosts (Fevre et al., “Human African Trypanosomiasis: Epidemiology and Control,” Adv. In Parasitol. 6: 167-222 (2006), which is hereby incorporated by reference in its entirety). In West and Central Africa T. b. gambiense is prevalent but is not found in cattle. Furthermore, some baboons (Papio papio) are not infected with T. b. gambiense, whereas other baboons (Papio hamadryas, used in this study) can be infected with T. b. gambiense but eventually clear the parasitemia, whether this is due to baboon TLF or not will likely require the generation of sustained transgenic expression of all three TLF components (Kageruka et al., “Infectivity of Trypanosoma (Trypanozoon) brucei gambiense for Baboons (Papio hamadryas, Papio papio),” Ann Soc Belg Med Trop 71: 39-46 (1991), which is hereby incorporated by reference in its entirety). The present invention can be used in the generation of resistant transgenic cattle has the potential to improve livestock productivity and reduce transmission of African trypanosomiasis caused by Trypanosoma spp.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A Trypanosome-resistant, non-human transgenic animal whose somatic and germ cells comprise a first nucleic acid transgene which encodes a recombinant apolipoprotein L-I polypeptide (apoL-I) having the amino acid sequence selected from the group consisting of SEQ ID NO: 1, SEQ ID NO: 3, SEQ ID NO: 4, and SEQ ID NO: 5, wherein the first nucleic acid transgene is operatively associated with at least one expression regulatory sequence.

2. The transgenic animal of claim 1, wherein the first nucleic acid transgene which encodes an apolipoprotein L-I polypeptide (apoL-I) has the amino acid sequence of SEQ ID NO: 5 from baboon (Papio hamadryas).

3. The transgenic animal of claim 1, wherein the transgenic animal has a stable plasma level of apolipoprotein L-I polypeptide throughout its sexual maturity.

4. The transgenic animal according to claim 1, wherein said expression regulatory sequence is a promoter sequence selected from the group consisting of a viral promoter, an apoA-I promoter, an apoL-I promoter, a hepatic enhancer of apoE, an intestinal enhancer of apoCIII, a ubiquitous promoter, a filament promoter, an MDR promoter, a CFTR promoter, factor promoter, a tissue-specific promoter, a promoter which is preferentially activated in dividing cells, a promoter which responds to a stimulus, a tetracycline-regulated transcriptional modulator, and a metallothionein promoter.

5. The transgenic animal according to claim 1, wherein the somatic and germ cells of the transgenic animal further comprise:
a second nucleic acid transgene encoding a haptoglobin related protein (Hpr) having an amino acid sequence of SEQ ID NO: 14, or SEQ ID NO: 16, wherein the Hpr encoding nucleic acid transgene is operatively associated with at least one expression regulatory sequence.

6. The transgenic animal of claim 5, wherein the second nucleic acid transgene encodes a haptoglobin related protein (Hpr) comprising the amino acid sequence of SEQ ID NO: 16 from baboon (Papio hamadryas).

7. The transgenic animal according to claim 1 wherein the animal is a mammal.

8. The transgenic animal according to claim 1, wherein the animal is selected from the group consisting of mouse, rat, pig, sheep, bovine animals, camel, and horse.

9. A method for producing a Trypanosome-resistant, non-human transgenic animal, said method comprising:
providing a first nucleic acid construct comprising a first nucleic acid molecule which encodes an apolipoprotein L-I polypeptide having the amino acid sequence selected from the group consisting of SEQ ID NO: 1, SEQ ID NO: 3, SEQ ID NO: 4, and SEQ ID NO: 5, wherein the first nucleic acid molecule is operably associated with at least one expression regulatory sequence;
introducing the first nucleic acid construct into a zygote of a non human animal;
transplanting said zygote into a pseudopregnant non-human female animal;
allowing said zygote to develop to term; and
identifying at least one transgenic offspring containing the first nucleic acid molecule.

10. The method according to claim 9, wherein the first nucleic acid molecule encodes apolipoprotein L-I polypeptide comprising the amino acid sequence of SEQ ID NO: 5 from baboon (Papio hamadryas).

11. The method according to claim 9, wherein the first nucleic acid construct further comprises a second nucleic acid molecule that encodes a haptoglobin binding protein (Hpr) having the amino acid sequence of SEQ ID NO: 14 or SEQ ID NO: 16, wherein the Hpr encoding nucleic acid sequence is operatively associated with at least one expression regulatory sequence.

12. The method according to claim 11, wherein the second nucleic acid molecule encodes haptoglobin binding protein comprising the amino acid sequence of SEQ ID NO: 16 from baboon (Papio hamadryas).

13. The method according to claim 9 further comprising:
providing a second nucleic acid construct comprising a nucleic acid molecule which encodes a haptoglobin binding protein (Hpr), having the amino acid sequence of SEQ ID NO: 14 or SEQ ID NO: 16, wherein the second nucleic acid molecule is operatively associated with at least one expression regulatory sequence and
introducing the second nucleic acid construct into the zygote of the non-human animal into which the first nucleic acid construct is introduced, and transplanted into the pseudopregnant non-human female animal.

14. The method according to claim 13, wherein the second nucleic acid molecule encodes haptoglobin binding protein comprising the amino acid sequence of SEQ ID NO: 16 from baboon (Papio hamadryas).

15. The method according to claim 9 further comprising:
breeding said offspring to form a non-human transgenic animal having the first nucleic acid molecule.

16. A method for producing a Trypanosome-resistant, non-human transgenic animal, said method comprising:
providing a first nucleic acid construct comprising a first nucleic acid molecule which encodes an apolipoprotein L-I polypeptide having the amino acid sequence selected from the group consisting of SEQ ID NO: 1, SEQ ID NO: 3, SEQ ID NO: 4, and SEQ ID NO: 5, wherein the first nucleic acid molecule is operably associated with at least one expression regulatory sequence;
introducing the first nucleic acid construct into an embryo of a non-human animal;
transplanting said embryo into a pseudopregnant non-human female animal;
allowing said embryo to develop to term; and
identifying at least one transgenic offspring containing the first nucleic acid molecule.

17. The method according to claim 16, wherein the first nucleic acid molecule encodes apolipoprotein L-I polypeptide comprising the amino acid sequence of SEQ ID NO: 5 from baboon (Papio hamadryas).

18. The method according to claim 16, wherein the first nucleic acid construct further comprises a second nucleic acid molecule that encodes a haptoglobin binding protein (Hpr) having the amino acid sequence of SEQ ID NO: 14 or SEQ ID NO: 16, wherein the Hpr encoding nucleic acid sequence is operatively associated with at least one expression regulatory sequence.

19. The method according to claim 18, wherein the second nucleic acid molecule encodes haptoglobin binding protein comprising the amino acid sequence of SEQ ID NO: 16 from baboon (Papio hamadryas).

20. The method according to claim 16 further comprising:
providing a second nucleic acid construct comprising a second nucleic acid molecule which encodes a haptoglobin binding protein (Hpr) having the amino acid sequence of SEQ ID NO: 14, or SEQ ID NO: 16, wherein the second nucleic acid molecule is operatively associated with at least one expression regulatory sequence and
introducing the second nucleic acid construct into the zygote of the non-human animal into which the first nucleic acid construct is introduced and transplanted into the pseudopregnant non-human female animal.

21. The method according to claim 20, wherein the second nucleic acid molecule encodes haptoglobin binding protein comprising the amino acid sequence of SEQ ID NO: 16 from baboon (Papio hamadryas).

22. The method according to claim 16, wherein said introducing of the first nucleic acid construct into the embryo comprises:
introducing an embryonic stem cell containing the first nucleic acid construct into the embryo.

23. The method according to claim 16, wherein said introducing of the first nucleic acid construct into the embryo comprises:
infecting the embryo with a virus containing the first nucleic acid construct.

24. The method according to claim 16 further comprising:
breeding said offspring to form a non-human transgenic animal having the first nucleic acid molecule.

25. A transgenic egg comprising:
a first nucleic acid transgene which encodes an apolipoprotein L-I polypeptide having the amino acid sequence selected from the group consisting of SEQ ID NO: 1, SEQ ID NO: 3, SEQ ID NO: 4, and SEQ ID NO: 5 and operatively associated with at least one expression regulatory sequence.

26. The method according to claim 25, wherein the first nucleic acid transgene encodes apolipoprotein L-I polypeptide comprising the amino acid sequence of SEQ ID NO: 5 from baboon (Papio hamadryas).

27. The transgenic egg according to claim 25 further comprising:
a second nucleic acid transgene sequence which encodes a haptoglobin binding protein having the amino acid sequence of SEQ ID NO: 14 or SEQ NO: 16 and operatively associated with at least one expression regulatory sequence.

28. The method according to claim 27, wherein the second nucleic acid transgene encodes haptoglobin binding protein comprising the amino acid sequence of SEQ ID NO: 16 from baboon (Papio hamadryas).

29. A transgenic sperm comprising:
a first nucleic acid transgene which encodes an apolipoprotein L-I polypeptide having the amino acid sequence selected from the group consisting of SEQ ID NO: 1, SEQ ID NO: 3, SEQ ID NO: 4, and SEQ ID NO: 5, and operatively associated with at least one expression regulatory sequence.

30. The method according to claim 29, wherein the first nucleic acid transgene encodes apolipoprotein L-I polypeptide comprising the amino acid sequence of SEQ ID NO: 5 from baboon (Papio hamadryas).

31. The transgenic sperm according to claim 29 further comprising:
a second nucleic acid transgene sequence which encodes a haptoglobin binding protein having the amino acid sequence of SEQ ID NO: 14 or SEQ ID NO: 16 and operatively associated with at least one expression regulatory sequence.

32. The method according to claim 31, wherein the second nucleic acid transgene encodes haptoglobin binding protein comprising the amino acid sequence of SEQ ID NO: 16 from baboon (Papio hamadryas).

33. A method of animal husbandry:
providing the transgenic animal of claim 1 and
raising the transgenic animal.

34. The method according to claim 33, wherein said raising the transgenic animal is carried out in an environment where the transgenic animal is susceptible to trypanosome infection.

35. The method according to claim 33, wherein the trypanosome is selected from the group consisting of Trypanosoma brucei rhodesiense, Trypanosoma brucei gambiense, Trypanosoma brucei brucei, Trypanosoma congolense, Trypanosoma suis, Trypanosoma evansi, Trypanosoma equinum, Trypanosoma equiperdum, Trypanosoma rangeli, Trypanosoma simiae, and Trypanosoma vivax.

36. The method according to claim 33, wherein the first nucleic acid molecule encodes apolipoprotein L-I polypeptide comprising the amino acid sequence of SEQ ID NO: 5 from baboon (Papio hamadryas).

37. The method according to claim 33, wherein the somatic and germ cells of the transgenic animal further comprise:
a second nucleic acid transgene encoding a haptoglobin related protein (Hpr) having an amino acid sequence of SEQ ID NO: 14 or SEQ ID NO: 16, wherein the Hpr encoding nucleic acid transgene is operatively associated with at least one expression regulatory sequence.

38. The method according to claim 37, wherein the second nucleic acid transgene encodes the haptoglobin binding protein comprising the amino acid sequence of SEQ ID NO: 16 from baboon (Papio hamadryas).

39. The method according to claim 33, wherein the animal is a mammal.

40. The method according to claim 33, wherein the animal is selected from the group consisting of mouse, rat, pig, sheep, bovine animals, camel, and horse.
